SEQUENCE LISTING (12 sequences)

SEQ ID NO: 1 - protein, 8 aa, Artificial Sequence
  primer sequence
NXCXHRGM

SEQ ID NO: 2 - protein, 7 aa, Artificial Sequence
  primer sequence
EQDDXEN

SEQ ID NO: 3 - DNA, 2120 bp, Burkholderia sp. LB400
  CDS (47)..(1426): bphA1
tattttttccgccctgccaagggcatttcaacggagacgttaaatcatgagttcagcaatcaaagaagtgcagggagcccctgtgaagtgggttaccaattggacgccggaggcgatccgggggttggtcgatcaggaaaaagggctgcttgatccacgcatctacgccgatcagagtctttatgagctggagcttgagcgggtttttggtcgctcttggctgttacttgggcacgagagtcatgtgcctgaaaccggggacttcctggccacttacatgggcgaagatccggtggttatggtgcgacagaaagacaagagcatcaaggtgttcctgaaccagtgccggcaccgcggcatgcgtatctgccgctcggacgccggcaacgccaaggctttcacctgcagctatcacggctgggcctacgacatcgccggcaagctggtgaacgtgccgttcgagaaggaagccttttgcgacaagaaagaaggcgactgcggctttgacaaggccgaatggggcccgctccaggcacgcgtggcaacctacaagggcctggtctttgccaactgggatgtgcaggcgccagacctggagacctacctcggtgacgcccgcccctatatggacgtcatgctggatcgcacgccggccgggactgtggccatcggcggcatgcagaagtgggtgattccgtgcaactggaagtttgccgccgagcagttctgcagtgacatgtaccacgccggcaccacgacgcacctgtccggcatcctggcgggcattccgccggaaatggacctctcccaggcgcagatacccaccaagggcaatcagttccgggccgcttggggcgggcacggctcgggctggtatgtcgacgagccgggctcactcctggcggtgatgggccccaaggtcacccagtactggaccgagggtccggctgccgagcttgcggaacagcgcctggggcacaccggcatgccggttcgacgcatggtcggccagcacatgacgatcttcccgacctgttcattcctgcccaccttcaacaacatccggatctggcacccgcgtggtcccaatgaaatcgaggtgtgggccttcaccctggtcgatgccgacgccccggcggagatcaaggaagaatatcgccggcacaacatccgcaacttctccgcaggcggcgtgtttgagcaggacgatggcgagaactgggtggagatccagaaggggctacgtgggtacaaggccaagagccagccgctcaatgcccagatgggcctgggtcggtcgcagaccggtcaccctgattttcctggcaacgtcggctacgtctacgccgaagaagcggcgcggggtatgtatcaccactggatgcgcatgatgtccgagcccagctgggccacgctcaagccctgatcaagacgcaatcgttagatctgtcaaccggaagaattcaacatggtgggctggacgtgcatgtgcagacggcgcgccgaggttccgtcccctgatatttacttggagataactgttatgacaaatccatccccgcattttttcaaaacatttgaatggccaagcaaggcggctggccttgagttgcagaacgagatcgagcagttctactaccgcgaagcgcagttgcttgaccaccgggcctacgaggcctggtttgccctgctggacaaagatatccactacttcatgccgctgcgcaccaatcgcatgatccgggagggcgagctggaatattccggcgaccaggatttagcccatttcgatgaaacccatgaaaccatgtacgggcgcatccgcaaggtgacctcggacgtgggctgggcggagaacccgccttcccgcacgcgccacctggtctccaacgtcatcgtcaaggagacggccacgccggataccttcgaggtcaattccgcattcatcctgtaccgcaatcggcttgagcgccaggtcgacatcttcgcgggcgaacgccgggacgtgctgcgccgcgccgacaacaaccttggtttcagcatcgccaagcgcaccatcctgctcgacgccagtaccttgctgtcgaacaacctgagcatgttcttctagcccagcacgc

SEQ ID NO: 4 - protein, 459 aa, Burkholderia sp. LB400
MSSAIKEVQGAPVKWVTNWTPEAIRGLVDQEKGLLDPRIYADQSLYELELERVFGRSWLLLGHESHVPETGDFLATYMGEDPVVMVRQKDKSIKVFLNQCRHRGMRICRSDAGNAKAFTCSYHGWAYDIAGKLVNVPFEKEAFCDKKEGDCGFDKAEWGPLQARVATYKGLVFANWDVQAPDLETYLGDARPYMDVMLDRTPAGTVAIGGMQKWVIPCNWKFAAEQFCSDMYHAGTTTHLSGILAGIPPEMDLSQAQIPTKGNQFRAAWGGHGSGWYVDEPGSLLAVMGPKVTQYWTEGPAAELAEQRLGHTGMPVRRMVGQHMTIFPTCSFLPTFNNIRIWHPRGPNEIEVWAFTLVDADAPAEIKEEYRRHNIRNFSAGGVFEQDDGENWVEIQKGLRGYKAKSQPLNAQMGLGRSQTGHPDFPGNVGYVYAEEAARGMYHHWMRMMSEPSWATLKP

SEQ ID NO: 5 - protein, 213 aa, Burkholderia sp. LB400
MVGWTCMCRRRAEVPSPDIYLEITVMTNPSPHFFKTFEWPSKAAGLELQNEIEQFYYREAQLLDHRAYEAWFALLDKDIHYFMPLRTNRMIREGELEYSGDQDLAHFDETHETMYGRIRKVTSDVGWAENPPSRTRHLVSNVIVKETATPDTFEVNSAFILYRNRLERQVDIFAGERRDVLRRADNNLGFSIAKRTILLDASTLLSNNLSMFF

SEQ ID NO: 6 - DNA, 1600 bp, Burkholderia sp. LB400
  CDS (42)..(371): bphA3
cccaggcgatttaacccttttaactaattacaagaagcgttatgaaatttaccagagtttgtgatcgaagagatgtgcccgaaggcgaagccctgaaggtcgaaagtggaggcacctccgtcgcgattttcaatgtggatggcgagctgttcgcaacacaggaccgctgcacccacggcgactggtccctgtccgatggcggctatcttgaaggtgacgtggtggaatgctcactgcacatggggaagttttgcgttcgcacgggcaaggtcaaatcaccgccgccctgtgaggcactgaagatatttccgatccgcatcgaagacaatgacgtgctggtcgacttcgaagccgggtatctggcgccatgatcgacaccatcgccatcatcggcgccggcctggccggttcgacggctgcgcgcgcactgcgcgcccagggatacgaggggcgcatccacctgctcggggatgagtcgcatcaggcctatgaccggaccacgctgtccaagacggtgctggcgggcgagcagcccgagccgcctgcaatcctggacagcgcctggtacgcatcggcccatgtggatgtccagctcgggcgacgggtgagttgcctggatctggccaaccgccagattcagtttgaatcgggcgccccgctggcctacgaccggctgctgctggccaccggcgcgcgcgcccggcgcatggcgattcggggtggcgacctggcaggcatccataccttgcgagacctcgccgacagccaggcgctgcggcaggcgctgcaaccgggccagtcgctggtcatcgtcggcggaggcctgatcggttgcgaggtggcgaccaccgcccgcaagctgagtgtccatgtcacgattctggaagccggcgacgagttgctggtgcgcgtgctgggtcaccggaccggggcatggtgtcgggccgaactggaacgcatgggtgtccgcgtggagcgcaatgcacaggccgcgcgcttcgaaggccaggggcaggtgcgcgccgtgatctgcgccgacgggcgccgggtgcccgccgatgtggtcttggtcagcattggcgccgagccggcggacgagctggcccgtgccgctggcatcgcctgcgcgcgcggcgtgctggtcgacgccaccggcgccacctcgtgtccagaggtgttcgccgccggtgacgtcgccgcctggccgctgcgtcaagggggccagcgctcgctggagacctacctgaacagccagatggaggccgaaatcgcggccagcgccatgttgagtcagcccgtgccggcgccccaggtgccgacctcgtggacggagattgcaggccaccgcatccagatgattggcgatgccgaagggcccggcgagatcgtcgtacgcggcgacgcccagagcggccagccaatcgtgttgctcaggctgcttgatggctgcgtcgaggccgcgacggcgatcaatgccaccagggaattttctgtggcgacccgactggtcggcacccgggtttctgtttccgccgagcaactgcaggacgtcggctcgaacctgcgggatttactcaaagccaaaccgaattgatgcgca

SEQ ID NO: 7 - protein, 109 aa, Burkholderia sp. LB400
MKFTRVCDRRDVPEGEALKVESGGTSVAIFNVDGELFATQDRCTHGDWSLSDGGYLEGDVVECSLHMGKFCVRTGKVKSPPPCEALKIFPIRIEDNDVLVDFEAGYLAP

SEQ ID NO: 8 - protein, 408 aa, Burkholderia sp. LB400
  MISC_FEATURE: BphA4
MIDTIAIIGAGLAGSTAARALRAQGYEGRIHLLGDESHQAYDRTTLSKTVLAGEQPEPPAILDSAWYASAHVDVQLGRRVSCLDLANRQIQFESGAPLAYDRLLLATGARARRMAIRGGDLAGIHTLRDLADSQALRQALQPGQSLVIVGGGLIGCEVATTARKLSVHVTILEAGDELLVRVLGHRTGAWCRAELERMGVRVERNAQAARFEGQGQVRAVICADGRRVPADVVLVSIGAEPADELARAAGIACARGVLVDATGATSCPEVFAAGDVAAWPLRQGGQRSLETYLNSQMEAEIAASAMLSQPVPAPQVPTSWTEIAGHRIQMIGDAEGPGEIVVRGDAQSGQPIVLLRLLDGCVEAATAINATREFSVATRLVGTRVSVSAEQLQDVGSNLRDLLKAKPN

SEQ ID NO: 9 - DNA, 33 bp, Artificial Sequence
  PCR primer 1 containing XbaI site
atgcattctagatattttttccgccctgccaag

SEQ ID NO: 10 - DNA, 33 bp, Artificial Sequence
  PCR primer 2 containing NcoI site
atgcatccatggcgtgctgggctagaagaacat

SEQ ID NO: 11 - DNA, 34 bp, Artificial Sequence
  PCR primer 3 containing NcoI site
atgcatccatggcccaggcgatttaaccctttta

SEQ ID NO: 12 - DNA, 31 bp, Artificial Sequence
  PCR primer 4 containing NdeI site
atgcatcatatgcgcatcaattcggtttggc

(19) United States
(12) Patent Application Publication
(10) Pub. No.: US 2016/0279456 A1
(43) Pub. Date: Sep. 29, 2016

(54) POLYCHLORINATED BIPHENYL DETOXIFYING COMPLEX COMPOSITION AND METHOD FOR MANUFACTURING SAME

(71) Applicant: NATIONAL UNIVERSITY CORPORATION YAMAGATA UNIVERSITY, Yamagata-shi, Yamagata (JP)

(72) Inventors: Tomijiro HARA, Yamagata-shi (JP); Yumiko TAKATSUKA, Yamagata-shi (JP); Tokio NIIKUNI, Yamagata-shi (JP)

(73) Assignee: NATIONAL UNIVERSITY CORPORATION YAMAGATA UNIVERSITY, Yamagata-shi, Yamagata (JP)

(21) Appl. No.: 14/778,388

(22) PCT Filed: Mar. 20, 2014
(86) PCT No.: PCT/JP2014/057865

§ 371 Date: Jun. 9, 2016

(30) Foreign Application Priority Data
Mar. 21, 2013 (JP) 2013-058850

(51) Int. Cl. A62D 3/02; C12N 1/20; C12N 9/02

(52) U.S. Cl. CPC A62D 3/02; C12N 9/0071; C12N 1/20; C12Y 114/12018; A62D 2101/22

(57) ABSTRACT

Provided is a polychlorinated biphenyl-decomposing composition obtained according to a method comprising respectively culturing at least one main microbial strain belonging to Comamonas species and having biphenyl dioxygenase, and at least one complementary microbial strain selected from the group consisting of Pseudomonas species, Achromobacter species, Rhodococcus species and Stenotrophomonas species and having biphenyl dioxygenase, and mixing at least two types of microbial cells recovered from each of the culture media. A composition containing these compounded microorganisms is useful for efficiently decomposing or detoxifying comparatively low concentrations of PCBs present in large amounts in waste products contaminated with polychlorinated biphenyls.

### TECHNICAL FIELD

The present invention relates to a composition that effectively decomposes polychlorinated biphenyls (which may also be referred to as “PCBs”) by incorporating and compounding a plurality of microorganisms having different properties capable of decomposing polychlorinated biphenyls, and to a method for producing that composition. More particularly, the present invention relates to a composition obtained by adding and compounding a microorganism belonging to Pseudomonas species, Rhodococcus species, Achromobacter species or Stenotrophomonas species with a microorganism belonging to Comamonas species, a production method thereof, and a method for efficiently decomposing polychlorinated biphenyls using that composition.

### BACKGROUND ART

Polychlorinated biphenyls refer to the generic term for compounds in which one or more hydrogen atoms of a biphenyl have been substituted with chlorine atoms. Although there are numerous isomers depending on the number and locations of the substituted chlorine atoms, they are theoretically known to be categorized into 209 types. Since PCBs are stable with respect to metal and have superior insulating properties, incombustibility, lipid solubility, plasticity and the like, they have been used in an extremely diverse range of product fields, such as electrical products, heating media, insulating oil and paint, and carbonless copy paper solvents. However, since they are highly toxic to the body, easily accumulate in organs and fatty tissue, are carcinogenic, and cause accompanying skin disorders, internal disorders, hormone abnormalities and the like, their use is prohibited not only domestically, but internationally as well. Since PCBs are chemically stable enabling them to persist for a long period of time without undergoing spontaneous decomposition, they present the significant problem of having serious effects on not only humans, but also on various forms of life present on the entire planet.

Roughly 55,000 tons of PCBs have been estimated to have previously been imported, manufactured and sold in Japan (see, for example, Non-Patent Document 1). Although a program has been implemented for detoxifying PCBs by 2016 following their prohibition and mandatory storage by users, since the cost of this detoxification is high and exposure has been recognized to pose a threat to workers, the program is currently not proceeding as scheduled. In addition, it has also been determined that, differing from previously detected PCB concentrations, trace amounts of PCBs on the order of about several tens of milligrams per kilogram have been detected in the insulating oil of numerous types of electrical equipment despite PCBs not having been previously used in that equipment. On the basis of these findings, a portion of the enforcement ordinance of the Special Measures Law relating to the proper treatment of polychlorinated biphenyl waste was revised on Dec. 12, 2012, and the deadline for decomposition treatment of PCBs was newly established to be on Mar. 31, 2027. Accurate quantities of this trace PCB-contaminated oil relating to use or storage are unable to be determined. More recently, however, the problem of PCBs being unintentionally produced as by-products in organic pigments has occurred, and a notification was issued indicating that manufacturers must recover all such pigment. In view of these social circumstances as well, it is clear that there is a need to implement continuing countermeasures for decomposition or detoxification of PCBs in the future. Known examples of methods used to decompose PCBs include conventional incineration as well as dechlorination and decomposition, hydrothermal oxidation decomposition, reduction thermochemical decomposition using a hydrogen donor and photodecomposition by ultraviolet irradiation and the like. Among these, since ultraviolet irradiation dechlorinates PCBs by dissolving in a polar organic solvent and irradiating the solution with ultraviolet light followed by detoxifying residual PCBs by biological treatment or catalytic treatment, it is possible for toxic PCBs to be catabolized by living organisms in the form of microorganisms with a high degree of safety as a result of being able to carry out treatment at normal temperature and normal pressure and the products of this decomposition are presumed to be highly safe, thereby making this advantageous in comparison with chemical treatment and the like.

For example, the method described in Patent Document 1 consists of initially carrying out dechlorination treatment by exposing PCBs to ultraviolet light followed by decomposing with microorganisms of a large-scale fermentation plant. However, this treatment method has a problem with respect to treatment of large amounts of oil contaminated with a high concentration of PCBs up to as much as 60% (w/v) to 80% (w/v) all at once, and presents difficulties in that PCB concentration must be adjusted by adding a large amount of medium in the microbial treatment step following the ultraviolet exposure step, while also requiring that microbial culturing and growth and PCB decomposition be carried out simultaneously.

Examples of microorganisms that have been previously reported to be organisms capable of decomposing PCBs include Pseudomonas species strain KKS102 (see, for example, Patent Documents 2 and 3), Comamonas testosteroni strain TK102 (Non-Patent Document 2) and Rhodococcus opacus strain TSP203 (Non-Patent Document 3). These microorganisms express a group of enzymes, including biphenyl dioxygenase (BphA), involved in a biphenyl decomposition pathway. In addition, a decomposition and elimination method has also been proposed in which a complex microbial circulation cycle is induced in PCB-decomposing microorganisms by complex fermentation, and decomposing microorganisms and decomposing enzymes against refractory PCBs or dioxins are generated and expressed (see, for example, Patent Document 4).

However, as a result of individually culturing these PCB-decomposing microorganisms and carefully examining the decomposition properties against individual polychlorinated biphenyl isomers, a complex microbial preparation incorporating a combination of a plurality of microorganisms optimal for PCB decomposition has yet to be known.

### PRIOR ART DOCUMENTS

### Patent Documents

Patent Document 1: Japanese Unexamined Patent Publication No. 2001-46547
Patent Document 2: Japanese Patent No. 2706718
Patent Document 3: Japanese Patent No. 2967950
Patent Document 4: Japanese Unexamined Patent Publication No. 2004-009046

### Non-Patent Documents

Non-Patent Document 1: “PCBs: The Negative Legacy of the 20th Century”, Chunichi Shimbun Newspaper, Sunday Edition, The Chunichi Shimbun Co., Ltd., Nov. 18, 2012
Non-Patent Document 2: Shimura, M. et al., Journal of Fermentation and Bioengineering, Vol. 81, No. 6, pp. 573-576, 1996
Non-Patent Document 3: Mukerjee-Dhar, G., Shimura, M. and Kimbara, K., Enzyme and Microbial Technology, Vol. 23, pp. 34-41, 1996
Non-Patent Document 4: Kimbara, K., et al., Agric. Biol. Chem., 52(11), pp. 2885-2891, 1988

### DISCLOSURE OF THE INVENTION

### Problems to be Solved by the Invention

PCBs refer to the generic term for compounds theoretically having a large number of 209 isomers, and it has been previously determined by numerous researchers, including the inventors of the present invention, that decomposing this large number of chlorinated biphenyl isomers all at once is difficult using only isolated PCB-decomposing microorganisms.

Therefore, an object of the present invention is to further improve the decomposition rate of all of the numerous types of the entire PCB isomer group, which are unable to be decomposed with PCB-decomposing microorganisms alone, by incorporating and compounding at least two types of microorganisms specified in terms of microbial taxonomy.

### Means for Solving the Problems

As a result of using soil and samples collected in the city of Yonezawa and its surrounding area to screen for biphenyl-decomposing microorganisms in a synthetic medium having biphenyl as its sole carbon source, the inventors of the present invention isolated more than 100 strains of microorganisms including Comamonas species, Achromobacter species, Pseudomonas species, Rhodococcus species and Stenotrophomonas species. As a result of investigating the decomposition properties of all of these microorganisms against individual polychlorinated biphenyl isomers, the decomposition properties of these microorganisms became clear, such as the same isomers being decomposed by individual genii or only specific isomers being characteristically decomposed by certain genii. Namely, it was found that a novel composition that has acquired a high decomposition capacity unable to be obtained with microbial species alone can be created by respectively compounding microorganism species having different decomposition properties against types of polychlorinated biphenyl isomers to form an artificially composed composition, thereby leading to completion of the present invention.

The microbial species used in the present invention consist of Comamonas species, Pseudomonas species, Achromobacter species, Rhodococcus species and Stenotrophomonas species, and each of these microorganisms has activity that decomposes polychlorinated biphenyls by assimilating biphenyls with a series of biphenyl-decomposing enzymes, including biphenyl dioxygenase (by metabolizing biphenyls within their cells and using as a source of nutrients). However, there are some microorganisms respectively belonging to Stenotrophomonas species and Achromobacter species that do not demonstrate decomposition activity against polychlorinated biphenyls when each is present alone, but demonstrate decomposition activity against biphenyls and polychlorinated biphenyls symbiotically, although the mechanism responsible for this is unknown. Thus, in one aspect of the present invention, a method for producing a polychlorinated biphenyl-decomposing composition comprising respectively culturing at least two or more types of PCB-decomposing microbial strains selected from microorganisms that belong to these specific genii and have biphenyl dioxygenase activity, and mixing at least two or more types of microorganisms recovered from each culture.

In one embodiment of the present invention, a PCB-decomposing microorganism belonging to Comamonas species is selected as the main microbial strain. In addition, a complementary microbial strain compounded therewith is at least one or more types of PCB-decomposing microorganisms selected from the group consisting of Pseudomonas species, Achromobacter species, Rhodococcus species and Stenotrophomonas species. The production method comprises a step in which each of these microorganisms is cultured by aeration-agitation culturing in medium containing biphenyl for the carbon source thereof, and a step for mixing at least two or more types of microorganisms recovered from each of the cultures.

Preferable examples of compounded microbial species belonging to the aforementioned genus Comamonas include Comamonas testosteroni strains YU14-111, YAZ1 and YAZ2, and one of these microbial strains may be used or two or more of these microbial strains may be used after mixing. Pseudomonas species strain YAZ51 of Pseudomonas, Achromobacter species strain YAZ52 of the genus Achromobacter, Rhodococcus species YAZ54 of the genus Rhodococcus, Stenotrophomonas species of the genus Stenotrophomonas, and Stenotrophomonas species/Achromobacter species strain YAZ21, which is symbiotic with Achromobacter species, of the genus Achromobacter, are preferable. Compounding refers to the obtaining of a composition that incorporates these microorganisms.

In another aspect of the present invention, a polychlorinated biphenyl-decomposing composition is provided that is produced by compounding according to the aforementioned production method or can be obtained according to the aforementioned production method. This polychlorinated biphenyl-decomposing composition is able to incorporate still other microbial cells, and such microorganisms are preferably microorganisms expressing a biphenyl dioxygenase complex having biphenyl-3,4-dioxygenase activity against at least one type of polychlorinated biphenyl. The aforementioned biphenyl dioxygenase complex preferably contains a BphA complex derived from Burkholderia xenovorans strain LB400 or a homologous protein that has sequence homology with each of the aforementioned amino acid sequences of 90% or more, and a complex thereof has polychlorinated biphenyl decomposition activity.

In a different aspect, the present invention provides a method for decomposing or detoxifying PCBs by contacting the aforementioned polychlorinated biphenyl-decomposing composition with PCBs. The aforementioned composition is preferably a composition in which microbial cells obtained by culturing the aforementioned microorganisms are frozen after their preliminary incorporation, and are either thawed at the time of use or are in the form of a dry composition that contains the plurality of types of microbial cells and an excipient. The dried composition in this case can be dried by freeze-drying or dry spraying and the like. In one embodiment, a method for decomposing polychlorinated biphenyls is provided that comprises a step for mixing and emulsifying an oily component containing PCBs, the polychlorinated biphenyl-decomposing composition obtained according to the aforementioned production method, and depending on the case, an aqueous medium containing a surfactant, and a step for aerating and agitating the aforementioned emulsion.

### Effects of the Invention

Since a composition obtained according to the production method of the present invention demonstrates high PCB decomposition activity in the state of wet cells or in the state of dry cells, it can be used as a composition obtained by compounding microorganisms, and is able to decompose PCBs more efficiently in comparison with methods of the prior art. In addition, in a preferred embodiment, since the composition can be stored in the state of dry microbial cells, putrefaction and deterioration that occur in the state of wet microbial cells are prevented, thereby enhancing transportability and storageability. Since the composition can be easily added during actual PCB decomposition work, workability can be improved and PCBs can be decomposed with favorable reproducibility.

### BRIEF DESCRIPTION OF THE DRAWINGS

FIG. 1 indicates the results of amplifying a BphA1 gene fragment using as template DNA extracted from Comamonas testosteroni strain YAZ2, Pseudomonas sp. strain YAZ51, Achromobacter sp. strain YAZ52, Rhodococcus sp. strain YAZ54 and Stenotrophomonas sp./Achromobacter sp. strain YAZ21.

FIG. 2 is a graph indicating the PCB decomposition rates of compositions obtained by arbitrarily incorporating Pseudomonas sp. strain YAZ51, Achromobacter sp. strain YAZ52 or Stenotrophomonas sp./Achromobacter sp. symbiotic strain YAZ21 with the Comamonas testosteroni strain YAZ2 according to the present invention.

FIG. 3 indicates the structure of a plasmid pEA1A2A3A4(LB400) expressing a biphenyl dioxygenase complex that is derived from Burkholderia xenovorans strain LB400.

FIG. 4(A) indicates the results of analyzing total protein expressed by culturing Escherichia coli strain BL21(DE3) transformed with vector pEA1A2A3A4(LB400) or vector pET-15b by SDS-polyacrylamide electrophoresis (SDS-PAGE) at a gel concentration of 15%. FIG. 4(B) indicates results depicting the decomposition rates after contacting a suspension of microbial cells, containing a biphenyl dioxygenase complex expressed in Escherichia coli strain BL21(DE3) transformed with vector pAE1A2A3A4(LB400) or vector pET-15b (concentration OD₆₆₀=10), with Kanechlor KC-300 (5 ppm) and allowing to react for 24 hours. Escherichia coli strain BL21(DE3) transformed only with vector pET-15b was used as a control in the same manner as (A).

FIG. 5 indicates results depicting the growth curves of microbial strains expressing BphA1A2A3A4(LB400) during addition of IPTG at a final concentration of 0.1 mM (A) or 0.2 mM (B) using expression induction conditions in Escherichia coli strain BL21(DE3) transformed with pEA3A2A3A4(LB400), and the PCB decomposition rates attributable to microbial cells harvested over time.

FIG. 6 indicates the results of investigating the relationship between culture broth turbidity and PCB decomposition rate during addition of IPTG using expression induction conditions in Escherichia coli strain BL21(DE3) transformed with pEA1A2A3A4(LB400).

FIG. 7 indicates the results of GM-MS analysis of residual PCB isomers when Kanechlor KC-300 was decomposed using bacterial cells expressing a biphenyl dioxygenase complex.

FIG. 8 indicates the results of investigating the relationship between PCB decomposition rate and the compounding (incorporation) ratio of two types of microbial cells expressing a biphenyl dioxygenase complex.

FIG. 9 indicates the results of a more detailed investigation of the relationship between PCB decomposition rate and the compounding (incorporation) ratio of two types of microbial cells expressing a biphenyl dioxygenase complex.

FIG. 10 is a graph indicating time-based changes in the amount of residual PCBs when PCB-contaminated insulating oil was decomposed using the PCB decomposition apparatus of Reference Example 6.

### BEST MODE FOR CARRYING OUT THE INVENTION

The present applicant had previously isolated a novel microorganism in the form of Comamonas testosteroni strain YU14-111 (to also be referred to as “strain YU14-111”) as a result of using samples collected from soil collected in the city of Yonezawa and its surrounding area as well as from activated sludge of a water treatment plant, and using those samples to screen for biphenyl-decomposing microorganisms in synthetic medium that uses biphenyls for the carbon source, and has applied for patent (see Japanese Patent Application No. 2012-046270 and its unexamined publication in the form of Japanese Unexamined Patent Publication No. 2013-179890). A method for decomposing PCBs using this novel microorganism demonstrated considerable efficacy in comparison with methods of the prior art, and although the decomposition rate thereof reached 81.7±1.36% in the case of Kanechlor KC-300, undecomposed PCBs still remained. Therefore, as a result of further studies, it was found that the PCB decomposition rate can be further improved by using a composition obtained by incorporating and compounding at least two or more types of microorganisms belonging to taxonomically specific genii. The PCB-decomposing composition of the present invention is characterized by comprising at least one microorganism belonging to Comamonas species that exhibits biphenyl dioxygenase activity as the main microbial strain, and at least one complementary microbial strain selected from the group consisting of Pseudomonas species, Achromobacter species, Rhodococcus species and Stenotrophomonas species that exhibits biphenyl dioxygenase, and preliminarily culturing each of these microbial strains. At this time, by using biphenyl as a metabolism-inducing substance while providing an adequate supply of oxygen by aeration and stirring, the expression of a series of metabolizing enzymes, including biphenyl dioxygenase, is induced that are involved in the decomposition of PCBs, and a composition having a high level of PCB decomposition activity can be produced.

[Screening Method for Biphenyl-Assimilating Microorganisms]

The types of microorganisms able to be used to produce the polychlorinated biphenyl-decomposing composition of the present invention are only required to be microorganisms that have a gene of a biphenyl-decomposing (also referred to as PCB-decomposing) group of enzymes in a genome or plasmid and have a rapid growth rate, and such microorganisms are normally found by repeatedly screening microorganisms capable of growing using biphenyls as the only carbon source. Although there are numerous bacteria in nature that produce biphenyl-decomposing enzymes, such as those belonging to Pseudomonas species, Comamonas species, Burkholderia species, Sphingomonas species, Rhodococcus species or Ralstonia species, selecting microorganisms that rapidly produce meta cleavage products (such as 2-hydroxy-6-oxo-6-phenylhexa-2,4-dienoate), which exhibit a yellow-orange color and are used as an indicator of biphenyl decomposition activity at the screening stage of biphenyl-assimilating microorganisms, and have a rapid growth rate can be expected to enable acquisition of microorganisms having superior PCB decomposition activity.

Alternatively, a gene cluster having further improved PCB decomposition activity can be produced by cloning a biphenyl-decomposing enzyme gene from the resulting microorganism and introducing a mutation therein by a method such as site-directed mutagenesis. Furthermore, a known means or method complying therewith, such as the Kunkel method or gapped duplex method, can be used to introduce a mutation into a gene. In addition, a mutation can be introduced into a gene or a chimeric gene can be constructed by a technique such as error-prone PCR or DNA shuffling, and for example, Chen, K. and Arnold, F. H., 1993, Proc. Natl. Acad. Sci. USA, 90: 5618-5622 provides a description of error-prone PCR, while Kurtzman, A. L., Govindarajan, S., Vahle, K., Jones, J. T., Heinrichs, V. and Patten, P. A., Advances in directed protein evolution by recursive genetic recombination: Applications to therapeutic proteins, Curr. Opinion Biotechnol., 12, 361-370, 2001 provides a description of molecular evolutionary engineering techniques such as DNA shuffling or cassette PCR. A mutant gene produced by these techniques can be substituted with genomic DNA of the original microorganism or introduced into a host microorganism by cloning to plasmid DNA or cosmid DNA to enable the production of a novel microorganism. The PCB-decomposing microorganisms able to be used in the method of the present invention are thought to be able to be easily acquired by a person with ordinary skill in the art using such methods.

One of the microorganisms able to be used to produce the polychlorinated biphenyl-decomposing composition of the present invention was deposited by the present applicant in the Patent Microorganisms Depository of the National Institute of Technology and Evaluation (address: 2-5-8 Kazusakamatari, Kisarazu-shi, Chiba 292-0818, Japan) under accession number NITE P-1215 on Jan. 27, 2012 as Comamonas testosteroni strain YU14-111 (Lot Number YU14-11-03), after which it was transferred to an international deposit based on the Budapest Treaty on Mar. 5, 2014 and was assigned the reference number ABP-1215. Furthermore, 96 isolated strains of biphenyl-assimilating microorganisms other than Comamonas testosteroni strain YU14-111 have also been clearly determined to be microorganisms of the types shown in the following Table 1 following identification of genus and species by 16Sr DNA analysis, and these microorganisms were assigned serial numbers and stored in storage master cell packs. In addition, biphenyl dioxygenase retained by these microorganisms was able to be detected by, for example, amplifying a BphA1 gene fragment by PCR using a primer having a sequence complementary to a highly preserved region of BphA1 gene corresponding to the genomic information of biphenyl dioxygenase.

TABLE 1

| Genus | No. of Isolated Strains |
|---|---|
| Comamonas | 2 |
| Achromobacter | 30 |
| Pseudomonas | 15 |
| Rhodococcus | 30 |
| Stenotrophomonas | 18 |
| Acinetobacter | 1 |

[Culturing of Microorganisms Having Polychlorinated Biphenyl Decomposition Activity]

The following provides a detailed explanation of the culturing method and PCB decomposition method. A description is first provided of the method used to culture microorganisms having a high level of PCB decomposition activity. The medium is preferably a synthetic medium adjusted to pH 6.8 to 7.0 with reference to Non-Patent Document 4 and biphenyl is further added as a carbon source at 0.05% (w/v) to 0.1% (w/v). The biphenyl may also be added in the form of a solution obtained by preliminarily dissolving with dimethylsulfoxide. Three stages of pre-culturing are preferably carried out prior to final culturing in terms of suitably microbial growth. The procedure consists of placing 2 ml to 3 ml of medium containing 0.05% (w/v) to 0.1% (w/v) of biphenyl as previously described in a test tube and the like having a volume 10 times or more greater than the amount of medium, thawing microorganisms stored frozen at −80° C. or lower as quickly as possible and disseminating in glycerol adjusted to 15% (w/v) to 18% (w/v) to an OD₆₆₀ of 0.1 to 0.8 as the number of microbial cells, culturing to an OD₆₆₀ of 0.4 to 0.8 while shaking at 120 rpm at a temperature of 30° C. to 35° C., transferring the entire amount to 27 ml to 30 ml of synthetic medium similarly containing 0.05% (w/v) to 0.1% (w/v) of biphenyl contained in a flask and the like having a volume equal to 5 times or more the amount of medium, culturing to an OD₆₆₀ of 0.4 to 0.8 while shaking at 120 rpm at a temperature of 30° C. to 35° C., additionally transferring the entire amount to 270 ml to 300 ml of synthetic medium containing 0.05% (w/v) to 0.1% (w/v) of biphenyl contained in a flask and the like having a volume equal to 5 times or more the amount of medium, and culturing to an OD₆₅₀ of 0.4 to 0.8 while shaking at 120 rpm at a temperature of 30° C. to 35° C. Final culturing preferably uses an automated culture apparatus such as a fermenter that enables control of temperature and air or oxygen aeration, is equipped with a stirrer preferably of the turbine type for the shape of the stirrer blades, and enables control of the rotating speed thereof. Biphenyl is added to 2.7 L to 3 L of synthetic medium in the same manner as pre-culturing to 0.02% (w/v) to 0.05% (w/v) followed by adding the entire amount of the pre-cultured culture broth thereto. The stirrer rotating speed is adjusted to 400 rpm to 600 rpm, aeration is adjusted to 4 L/min to 5 L/min in the case of aeration, and the temperature is adjusted to 30° C. to 35° C. The use of a pressure discharge unit is even more preferable since it enhances oxygen concentration in the culture broth. In that case, discharge pressure is adjusted to 0.005 MPa to 0.01 MPa. Although the biphenyl serving as the carbon source is consumed as microbial growth progresses, it is preferable to continuously add biphenyl to 0.02% (w/v) to 0.05% (w/v), and biphenyl may be added in the form of a solution obtained by preliminarily dissolving with dimethylsulfoxide, thereby allowing the obtaining of a microorganisms that have acquired decomposition activity against more highly chlorinated PCBs. The pH of the culture broth during culturing is preferably within the range of 7.0 to 9.0 in order to have an effect on the yield of microorganisms, and pH is preferably adjusted as necessary by continuously adding an ammonium salt to 0.02% (w/v) to 0.05% (w/v), and ammonium sulfate is preferable for the ammonium salt. In addition, the ammonium sulfate is preferably added in the case the nitrogen source is consumed during culturing. Microorganisms having a high level of PCB decomposition activity are obtained at an OD₆₆₀ of 2.5 to 3.0 and wet yield of 15 to 20 g in the final culture broth.

Next, a description is provided of a culturing method allowing the obtaining of microorganisms having PCB decomposition activity both quickly and at a high recovery rate. Modified Terrific Broth consisting mainly of 2.4% yeast extract and 1.2% tryptone, containing disodium hydrogen phosphate and sodium dihydrogen phosphate at 70 mM each incorporated at a ratio of 6:4, and adjusted to pH of 6.8 to 7.0 after sterilizing in autoclave is preferably used for the medium. 0.02% (w/v) to 0.05% (w/v) of biphenyl is preferably added to the previously described modified Terrific Broth as an additional carbon source. Moreover, biphenyl may also be added in the form of a solution obtained by preliminarily dissolving with dimethylsulfoxide. Three stages of pre-culturing are preferably carried out prior to final culturing consisting of placing 2 ml to 3 ml of synthetic medium containing 0.05% (w/v) to 0.1% (w/v) of biphenyl in a test tube and the like having a volume 10 times or more greater than the amount of medium, quickly thawing microorganisms stored frozen at −80° C. or lower and disseminating in glycerol adjusted to 15% (w/v) to 18% (w/v) to an OD₆₆₀ of 0.1 to 0.9, culturing to an OD₆₆₀ of 0.6 to 0.8 while shaking at 120 rpm at a temperature of 30° C. to 35° C., transferring the entire amount to 27 ml to 30 ml of modified Terrific Mediaimilarly containing 0.05% (w/v) to 0.1% (w/v) of biphenyl contained in a flask and the like having a volume equal to about 5 times the amount of medium, culturing to an OD₆₆₀ of 0.6 to 0.9 while shaking at 120 rpm at a temperature of 30° C. to 35° C., additionally transferring the entire amount to 300 ml of modified Terrific Broth medium containing 0.05% (w/v) to 0.1% (w/v) of biphenyl contained in a flask and the like having a volume equal to about 5 times the amount of medium, and culturing to an OD₆₆₀ of 0.6 to 0.8 while shaking at 120 rpm at a temperature of 30° C. to 35° C. Final culturing preferably uses an automated culture apparatus such as a fermenter that enables control of temperature and air or oxygen aeration, is equipped with a stirrer preferably of the turbine type for the shape of the stirrer blades, and enables control of the rotating speed thereof. Biphenyl is added to 2.7 L to 3 L of synthetic medium in the same manner as pre-culturing to 0.02% (w/v) to 0.05% (w/v) followed by adding the entire amount of the pre-cultured culture broth thereto. The stirrer rotating speed is adjusted to 400 rpm to 600 rpm, aeration is adjusted to 4 L/min to 5 L/min in the case of aeration, and the temperature is adjusted to 30° C. to 35° C. The use of a pressure discharge unit is even more preferable since it enhances oxygen concentration in the culture broth. In that case, discharge pressure is adjusted to 0.005 MPa to 0.01 MPa. Although the biphenyl serving as the carbon source is consumed as microbial growth progresses, it is preferable to continuously add biphenyl to 0.02% (w/v) to 0.05% (w/v), and biphenyl may be added in the form of a solution obtained by preliminarily dissolving with dimethylsulfoxide, thereby ultimately allowing the obtaining of PCB-decomposing microorganisms at an OD₆₆₀ of 14 to 20 and wet yield of 100 to 150 g.

[Preparation of Polychlorinated Biphenyl-Decomposing Composition]

A culture containing PCB-decomposing microorganisms obtained in the manner described above can be directly formed into a powder, or microbial cells can be recovered by washing with water or a dispersion medium containing a surfactant and the like. The culture broth can be used as is after culturing or it can be concentrated under reduced pressure. In addition, the microbial cells can be harvested by centrifugal separation or a procedure such as density gradient centrifugation or biphasic separation is carried out to enable highly concentrated PCB-decomposing microorganisms to be isolated and recovered. A suspension may also be used in which PCB-decomposing microorganisms are dispersed in various types of dispersion media.

When producing a liquid composition, substances used for the purpose of improving storageability and stability can be added to the aforementioned culture. For example, a pH adjuster, preservative, antioxidant, stabilizer or buffer can be added.

In the case of a powdered composition, it is necessary to dry the microbial cells obtained from the aforementioned culture. Viable microorganisms can be powdered directly by a microbial cell drying method such as air-drying, freeze-drying or spray-drying. A protective agent such as skim milk is preferably used at this time. In addition, arbitrary substances such as an extender can be added for formulation. For example, examples of vehicles include sugars such as lactose, D-mannitol, D-sorbitol or sucrose, starches such as cornstarch or potato starch, and inorganic salts such as calcium phosphate, calcium sulfate or precipitated calcium carbonate, as well as arbitrary vehicles approved by the Feed Safety Law such as defatted rice bran, soybean powder, soybean curd refuse, peanut skin, bran, rice husk chaff, calcium carbonate, sugar, starch, brewer's yeast or flour. One type of these vehicles may be used alone or two or more types may be used in combination.

In a preferred embodiment of the present invention, microbial cells are preferably washed at least twice with physiological saline or 20 mM phosphate buffer, and sodium phosphate is preferably used for the phosphate salt. In addition, an excipient such as a sugar-alcohol may be added to the microbial cells, the sugar-alcohol is preferably alpha-, beta- or delta-mannitol, the microbial cells can then ultimately be stored in a freezer and the like set to a temperature of −20° C. to −80° C., and in the case of drying to form a powder, PCB-decomposing dry microbial cells are obtained that can be stored at a normal temperature of 15° C. to 25° C.

Microbial cells obtained according to each of the previously described methods are preferably in the form of a composition suitably compounded at an incorporation ratio so as to efficiently decompose PCBs, although the microbial cells may also be compounded at a suitable incorporation ratio so as to efficiently decompose PCBs in the state of wet microbial cells prior to drying, and may be in the form of a PCB-decomposing composite composition by adding an excipient such as a sugar-alcohol to the resulting complex.

The polychlorinated biphenyl-decomposing composition of the present invention contains at least two type or more types of microbial cells, the main microbial strain belongs to the Comamonas species and PCB-decomposing microorganisms are preferably used that demonstrate biphenyl dioxygenase activity. Since PCB-decomposing microorganisms belonging to the Comamonas species are Gram-negative bacteria, they demonstrate high resistance to numerous highly stimulatory organic compounds and drugs in comparison with Gram-positive microorganisms. This is because the composition of the cell wall in Gram-negative microorganisms has an outer membrane further to the outside of the peptidoglycan layer in common with Gram-positive microorganisms, thereby maintaining that resistance. In addition, since Comamonas species consist of fermenting bacteria in which bacterial cells aggressively undergo division, they have the property of enabling a large number of bacteria to be obtained by large-volume culturing. Namely, this composition is suitable for a method for decomposing a wide range of numerous PCB isomers using a large amount of bacteria highly resistant to PCBs, and decomposing the remaining PCB isomers with a small number of different bacteria.

At least one or more types of PCB-decomposing microorganisms selected from the group consisting of Pseudomonas species, Achromobacter species, Rhodococcus species and Stenotrophomonas species can be used as complementary microbial strains added to intensify the PCB-decomposing action of the main microbial strain. These complementary microbial strains exhibit a selected substrate specificity common to 2,3-diphenyl dioxygenase and demonstrate an even narrower range of substrate specificity with respect to PCB isomers. More specifically, these bacteria preferably selectively decompose 2,2′,4,4′-tetrachlorobiphenyl, 2,2′,4,5-tetrachlorobiphenyl and 2,2′,3,5′-tetrachlorobiphenyl.

The incorporation ratio between the main microbial strain and complementary bacterial strain is preferably 10:0.5 to 10:9.9 as the number of microbial cells converted on the basis of turbidity of the culture mediauch as absorbance at 660 nm (OD₆₆₀). Namely, the number of incorporated microbial cells of the complementary microbial strain does not exceed the number of incorporated microbial cells of the main microbial strain. The reason for this is thought to be to prevent enzyme molecules required during the PCB decomposition reaction from being unnecessarily consumed by the complementary microbial strain. Thus, an incorporation method that allows the obtaining of an efficient PCB decomposition rate requires adjustment of oxygen partial pressure and bacterial count (total amount or incorporation ratio) in the reaction solution, and a more preferable incorporation ratio of the main microbial strain to the complementary microbial strain is 10:1 to 10:6, more preferably 10:1 to 10:3 and most preferably about 10:1 to 10:1.5 in terms of the number of microbial cells converted on the basis of turbidity of the culture broth.

[Combinations of Microbial Cells Having Biphenyl-3,4-Dioxygenase Activity]

Biphenyl dioxygenases derived from microorganisms obtained by screening for biphenyl-assimilating microorganisms in nature according to the method described above all have biphenyl-2,3-dioxygenase activity. On the basis thereof, this enzyme activity is considered to be superior for efficiently decomposing PCBs and can be easily acquired by a person with ordinary skill in the art according to the method described in the present description. Here, “biphenyl-2,3-dioxygenase” refers to enzyme activity that enables an oxygenation reaction to be carried out on at least one type of polychlorinated biphenyl isomer at position 2 and position 3, respectively, of the biphenyl ring. For example, BphA derived from Comamonas testosteroni strain YAZ2 is known to have 2,3-dioxygenase activity against 2,4′,5-trichlorobiphenyl and 2,4,4′-trichlorobiphenyl.

On the other hand, according to findings of the inventors of the present invention, microorganisms are known that have biphenyl-3,4-dioxygenase activity, which although the presence thereof in nature in Japan is comparatively rare, is known to decompose a wide range of PCT isomers having a high degree of chlorine substitution (see, for example, Japanese Unexamined Patent Publication No. 2000-69967). Incorporating microbial cells in the manner of the composition of the present invention is thought to be useful for completely decomposing numerous types of PCB isomers. Here, “biphenyl-3,4-dioxygenase activity” refers to enzyme activity that enables an oxygenation reaction to be carried out on at least one type of polychlorinated biphenyl isomer at position 3 and position 4 of the biphenyl ring. For example, biphenyl dioxygenase derived from Burkholderia xenovorans strain LB400 is able to introduce an oxygen molecule into 2,5,4′-trichlorophenyl or 2,5,2′,5′-tetrachlorophenyl at positions 3 and 4 of a 2,5-dichlorophenyl ring.

In the present invention, microbial cells having biphenyl-3,4-dioxygenase activity are further preferably incorporated into the aforementioned PCB-decomposing composition based on such findings relating to the substrate specificity of biphenyl dioxygenase. BphA is composed of four subunits (BphA1, BphA2, BphA3 and BphA4), and the larger subunit (BphA1) is thought to be involved in the substrate specificity of a diatomic oxygenation reaction. Thus, in a preferred embodiment of the present invention, a composition is provided that comprises microbial cells further having biphenyl-3,4-dioxygenease activity in addition to the 2,3-dioxygenase activity possessed by the aforementioned main microbial strain and complementary microbial strain for which at least the structure of BphA1 differs, and as a result thereof, is a biphenyl dioxygenase (BphA) having different substrate specificity with respect to PCBs.

In the present invention, a preferable biphenyl dioxygenase having biphenyl-3,4-dioxygenase activity is a biphenyl dioxygenase derived from Burkholderia xenovorans strain LB400. The base sequence of the gene thereof is already known, and this enzyme can be easily expressed by recombinant DNA technology using the base sequence thereof. In one embodiment, a biphenyl dioxygenase complex derived from the aforementioned Burkholderia xenovorans strain LB400 comprises a protein composed of the amino acid sequences indicated in SEQ ID NOS: 4, 5, 7 and 8, or a homologous protein having sequence homology of 90% or more, preferably 95% or more and even more preferably 98% or more with each of the aforementioned amino acid sequences and in which complexes thereof have polychlorinated biphenyl decomposition activity. Namely, the homologous protein can be said to be, for example, that which has an amino acid sequence in which one or several amino acids have been deleted, substituted or added in each of the amino acid sequences of SEQ ID NOS: 4, 5, 7 and 8 within a range that does not impair biphenyl decomposition activity (and may also be referred to as a “homologue”). Here, several amino acid residues specifically refer to 20 or less, preferably 10 or less and more preferably 5 or less.

The percentage of homology (%) of an amino acid sequence is defined as, after having aligned a sequence with a reference polypeptide sequence, and if necessary, introducing a gap in order to achieve the maximum percentage of sequence homology, the percentage of amino acid residues in a complementary sequence that are identical to the amino acid residues in the reference polypeptide sequence in the case of not taking into consideration any conservative substitutions as a component of sequence homology. Alignment for determining the percentage of homology of an amino acid sequence can be achieved by using various methods within the scope of the art such as publicly available computer software in the manner of, for example, BLAST, BLAST-2, ALiGN or Megalign (DNASTAR) software. A person with ordinary skill in the art is able to determine those parameters suitable for aligning sequences (including any arbitrary algorithm required for achieving maximum alignment over the entire length of compared sequences).

Here, the results of conducting a homology search on the amino acid sequence of each enzyme that composes the BphA complex derived from Comamonas testosteroni strain YAZ2 using GENETYX-MAC sequence analysis software based on known amino acid sequences obtained from a database such as GenBank with respect to each of the enzymes derived from Burkholderia xenovorans strain LB400 are shown in the following Table 2.

TABLE 2

| | BphA1 | | BphA2 | | BphA3 | | BphA4 | |
|---|---|---|---|---|---|---|---|---|
| | No. of | Consistency | No. of | Consistency | No. of | Consistency | No. of | Consistency |
| | residues | (%) | residues | (%) | residues | (%) | residues | (%) |
| Comamonas testosteroni strain | 458 | 100 | 193 | 100 | 109 | 100 | 408 | 100 |
| YAZ2 and strain YU14-111 | | | | | | | | |
| Burkholderia xenovorans strain | 459 | 76 | 213 | 63 | 109 | 73 | 408 | 33 |
| LB400 | | | | | | | | |

Amino acid sequences of each enzyme used in the aforementioned homology search can be acquired from GenBank under the following accession numbers (indicated in order of BphA1, BphA2, BphA3 and BphA4). Strain YU-111: BAM05536, BAM05537, BAM05538, BphA4 not published; Strain LB-400: AAB63425, YP_556408, YU_556406, YP_556405. Furthermore, the amino acid sequences of each of the enzymes derived from Comamonas testosteroni strain YAZ2 were completely identical to those of strain YU14-111.

According to the results indicated in Table 2, sequence homology of enzymes derived from Burkholderia xenovorans strain LB400 were 80% or less with respect to the BphA complex derived from Comamonas testosteroni strain YAZ2, and are considered to have a certain degree of difference in terms of protein higher order structure based on this difference in amino acid sequences. Such a change in structure results in diversity of substrate specificity with respect to PCB isomers, and compounding this plurality of enzymes by incorporating in a complex is presumed to be useful in terms of efficiently decomposing PCB isomers.

Methods for introducing and expressing an artificially created gene in a microorganism based on a known amino acid sequence are known in the art. In one embodiment of the present invention, bphA1 gene derived from Burkholderia xenovorans strain LB400 is synthesized and used to transform a microorganism by using a recombinant vector in which this is functionally linked downstream from a promoter that acts within the cells of a host microorganism. In addition, in another embodiment, a microorganism is transformed using a recombinant vector containing bphA1 gene derived from Burkholderia xenovorans strain LB400 and another gene (bpHA2A3A4). In still another embodiment, a microorganism is co-transformed using a recombinant vector containing bphA1 gene and a recombinant vector containing another gene (bphA2A3A4). There are no particular limitations on the type of gene contained in the recombinant vector or the transformation sequence provided a microorganism is created that expresses the target BphA complex.

In the case of culturing host cells that have been transformed with a recombinant expression vector containing an inducible promoter in order to express an artificially created gene, an inducer may be added to the medium as necessary. For example, isopropyl-1-thio-β-D-galactoside (IPTG) can be added to the medium when culturing host cells transformed with an expression vector using lac promoter, while indole acrylic acid (IAA) can be added to the medium when culturing host cells transformed with an expression vector using trp promoter. Although there are no particular limitations on the culturing conditions, culturing is preferably carried out under conditions suitable for the host cells used in transformation.

The incorporation ratio of the main microbial strain to the complementary microbial strain in the polychlorinated biphenyl-decomposing composition of the present invention is preferably 10:0.5 to 10:9.9 as the number of microbial cells converted on the basis of turbidity of the culture mediauch as absorbance at 660 nm (OD₆₆₀). Namely, the number of incorporated microbial cells of the complementary microbial strain does not exceed the number of incorporated microbial cells of the main microbial strain. More preferably, the incorporation ratio of the main microbial strain to the complementary microbial strain is 10:1 to 10:6, more preferably 10:1 to 10:3 and most preferably about 10:1 to 10:1.5 in terms of the number of microbial cells converted on the basis of turbidity of the culture broth. Moreover, microbial cells having biphenyl-3,4-dioxygenase activity can be incorporated in a mixture of the aforementioned main microbial strain and complementary microbial strain at an arbitrary ratio, and in this case, there are no particular limitations on the incorporation ratio of the microbial cells having biphenyl-3,4-dioxygenase activity.

[Polychlorinated Biphenyl Decomposition Reaction]

The PCB decomposition method of the present invention is characterized by contacting PCBs with a composition containing microbial cells obtained by culturing the aforementioned microorganisms. In a preferred embodiment of the present invention, a composition obtained in this manner is able to demonstrate a high level of PCB decomposition activity when contacted with contaminated oil having a comparatively low concentration of PCBs.

PCBs targeted by the present invention include compounds in which chlorine atoms are substituted in a biphenyl compound, and the number of substituted chlorine atoms thereof is 1 to 10. The average number of substituted chlorine atoms is typically 2 to 6. In the present invention, at least one type selected from these PCBs can be used, and one type or a combination of two or more arbitrarily selected types can be used. In general, PCBs are not present in the form of single compounds, but rather are present in the form of mixtures of PCBs having different numbers of chlorine atoms and substitution positions. Thus, 209 types of isomers theoretically exist based on combinations of the numbers of chlorine atoms and substitution positions, and roughly 70 to 100 or more isomers are incorporated and available commercially.

Examples of PCBs able to be treated with the decomposition or detoxification method of the present invention characteristically include, but are not limited to, 3,4,4′,5-tetrachlorobiphenyl, 3,3′,4,4′-tetrachlorobiphenyl, 3,3′,4,4′,5-pentachlorobiphenyl, 2,3,3′,4,4′-pentachlorobiphenyl, 2,3,4,4′,5-pentachlorobiphenyl, 2,3′,4,4′,5-pentachlorobiphenyl and 2′,3,4,4′,5-pentachlorobiphenyl as well as 2,2′,4,4′-tetrachlorobiphenyl, 2,2′,4,5-tetrachlorobiphenyl and 2,2′,3,5′-tetrachlorobiphenyl.

PCBs are normally commercially available as mixtures of individual PCBs, and are used in capacitors and transformers. Specific examples thereof include Kanechlor KC-200 (bichlorinated biphenyl), KC-300 (trichlorinated biphenyl), KC-400 (tetrachlorinated biphenyl), KC-500 (pentachlorinated biphenyl), KC-600 (hexachlorinated biphenyl) and KC-100 (mixture of KC500 and trichlorobenzene at a ratio (weight ratio) of 60:40) manufactured and sold by Kanegafuchi Chemical Industry Co., Ltd., and Arochlor 1254 (54% chlorine) manufactured and sold by Mitsubishi Monsanto Chemical Co.

The PCB decomposition reaction according to the present invention comprises a step for mixing and emulsifying an oily component containing PCBs, the aforementioned PCB-decomposing composition and, depending on the case, a surfactant, and a step for aerating and agitating the aforementioned emulsion. PCBs targeted for decomposition can be contained at 0.05 mg/L to 1000 mg/L and preferably at about 1 mg/L to 100 mg/L based on the total amount of the emulsion, and the reaction can be carried out by adding the composition of the present invention at 0.2% by weight to 20% by weight and preferably at about 2% by weight to 12% by weight in the emulsion. In the case of not emulsifying, 0.005% of a surfactant such as Triton X-100 is added followed by further homogenizing by applying ultrasonic waves as necessary. Moreover, treatment for lowering the viscosity of the oil containing PCBs (such as alcoholization) may be carried out in advance in order to promote emulsification. Reaction conditions are such that temperature is adjusted to about 20° C. to 40° C., preferably 25° C. to 35° C. and even more preferably about 30° C., pH is preferably adjusted to pH 6 to 9, and treatment is preferably carried out for about 12 hours to 72 hours while stirring. This type of treatment can be carried out using a sealed reaction apparatus capable of stirring, or in other words, is preferably carried out using a compact, special-purpose apparatus. Reducing the size of the polychlorinated biphenyl decomposition reaction apparatus makes it possible to carry out treatment work directly even in a storage facility where trace amounts of PCBs are stored.

[Supply of Microbubbles]

In the present invention, the decomposition and detoxification of PCBs can be promoted by supplying microbubbles to the aforementioned polychlorinated biphenyl decomposition reaction system. Here, the term “microbubbles” refers to air bubbles having a diameter of about 1 mm or less and preferably 100 μm or less. Although air bubbles may be formed by supplying a gas such as oxygen or air from the outside or oxygen or air may be used after dissolving in an aqueous medium, in order to enhance the dissolved oxygen concentration of the aqueous medium, microbubbles are preferably generated while supplying oxygen gas from the outside. Since microbubbles have a large surface area per unit volume and an extremely slow ascent rate, a gas such as oxygen can be effectively dissolved in a liquid. In addition, microbubbles can be uniformly dispersed in a liquid by applying an electric charge, thereby promoting the emulsification of an oily component in an aqueous medium. Since microbubbles have a negative surface charge, they can be uniformly dispersed in an aqueous medium through interaction with microbial cells and the like typically having a positive surface charge.

A step for dispersing microbubbles in an aqueous medium may be carried out prior to mixing with an oily component containing PCBs or after mixing with an aqueous medium and oily component containing PCBs, followed by generating microbubbles in these mixtures. Examples of methods used to form microbubbles that may be used include a method consisting of expelling a gas through a pipe having micropores or a porous body in a liquid, a method for incorporating a gaseous phase in a liquid phase by utilizing shear force generated in a jet flow or rotational flow, and a method for forming fine air bubbles by vibrating a gas-liquid interface using ultrasonic waves.

In the PCB decomposition method of the present invention, an amount of oxygen that at least exceeds the saturated dissolved oxygen concentration in the aqueous medium is preferably contained in the aqueous medium, and microbubbles are therefore preferably generated by carrying out ultrasonic treatment while allowing oxygen gas to flow there through. These microbubbles are hereinafter referred to as oxygen microbubbles. Although saturated dissolved oxygen concentration varies according to such factors as air pressure, water temperature and dissolved salt concentration, the dissolved oxygen concentration in distilled water at atmospheric pressure and 30° C. is about 7.5 mg/L. In the method of the present invention, dissolved oxygen concentration in the aqueous medium at 30° C. at least has an initial concentration of about 8 mg/L, preferably 15 mg/L or more, and even more preferably 25 mg/L (ppm) or more. In one embodiment, in the case of filling oxygen microbubbles into the aqueous medium by the aforementioned ultrasonic wave generation method, the dissolved oxygen concentration thereof is about 28 mg/L in terms of the actual measured value. In general, oxygen dissolved in a highly concentrated state in an aqueous medium is thought to eventually decrease due to the property of attempting to achieve equilibrium with the oxygen concentration in the surrounding environment. Thus, in order to optimize a PCB decomposition reaction using the PCB-decomposing composition, it is preferable to maintain dissolved oxygen concentration that has increased to about 28 mg/L and continue to supply microbubbles either continuously or intermittently from a suitable microbubble generator.

### EXAMPLES

The following provides a detailed explanation of the microbial composite composition, including the production method of the present invention, by indicating examples and the like thereof. Furthermore, the present invention is not limited to these examples.

### Example 1

### Screening for Biphenyl-Assimilating Microorganisms

Synthetic medium (W medium) used for screening was composed as shown below with reference to the description of Non-Patent Document 4 in the same manner as the method indicated in Japanese Patent Application No. 2012-046270 filed by the present applicant and in its unexamined publication in the form of Japanese Unexamined Patent Publication No. 2013-179890.

TABLE 3

| Component | Content | |
|---|---|---|
| KH₂PO₄ | 1.7 | g/L |
| Na₂HPO₄ | 9.8 | g/L |
| (NH₄)₂SO₄ | 1.0 | g/L |
| MgSO₄•7H₂O | 0.1 | g/L |
| FeSO₄•7H₂O | 0.95 | mg/L |
| MgO | 10.75 | mg/L |
| CaCO₃ | 2.0 | mg/L |
| ZnSO₄•7H₂O | 1.44 | mg/L |
| CuSO₄•5H₂O | 0.25 | mg/L |
| CoSO₄•7H₂O | 0.28 | mg/L |
| H₃BO₃ | 0.06 | mg/L |
| conc. HCl | 51.3 | μl/L |

Medium having an indicated pH of 6.3 to 8.5 was prepared and biphenyl was added thereto as a carbon source at a final concentration of 0.1%. Samples measuring about one teaspoon were collected from soil in the city of Yonezawa and its surrounding area were added to the medium followed by shake-culturing for about 1 week to 1 month at 30° C. and 120 rpm. A procedure consisting of subculturing a portion of the culture broth in which concentration had increased in fresh medium followed by carrying out shake-culturing under the same conditions was repeated several times.

Microorganisms were isolated from enriched cultures in which growth of groups of biphenyl-assimilating microorganisms was observed. Namely, 20 μL of cultured broth from enriched cultures of biphenyl-assimilating microorganisms were applied to a synthetic medium plate, and biphenyl was supplied while evaporating by placing biphenyl powder on the cover of the inverted plate followed by culturing overnight or longer at 30° C. The colonies of various morphologies that grew were each streaked onto fresh synthetic medium plates followed by culturing while similarly supplying biphenyl by evaporation, and this was repeated until each colony became a single type of colony. The single colonies that grew were confirmed for the ability to assimilate biphenyl by inoculating into liquid synthetic medium containing 0.1% biphenyl, and the morphology of the colonies was observed by re-inoculating into the synthetic medium master plate in which biphenyl powder had been placed on the cover. Finally, in addition to confirming the ability to assimilate biphenyl of microorganisms re-isolated from the master plate in the form of single colonies, species were identified by 16S rDNA sequence analysis. Microbial strains isolated in this manner were named strain YAZ_ (where, _ indicates a number represented with an arbitrary Arabic numeral), and glycerol stocks were prepared and stored in a freezer set to −80° C.

### Example 2

### Detection of Biphenyl Dioxygenase Genes of Consisting of Strain YAZ2, YAZ21, YAZ51, YAZ52 and YAZ54

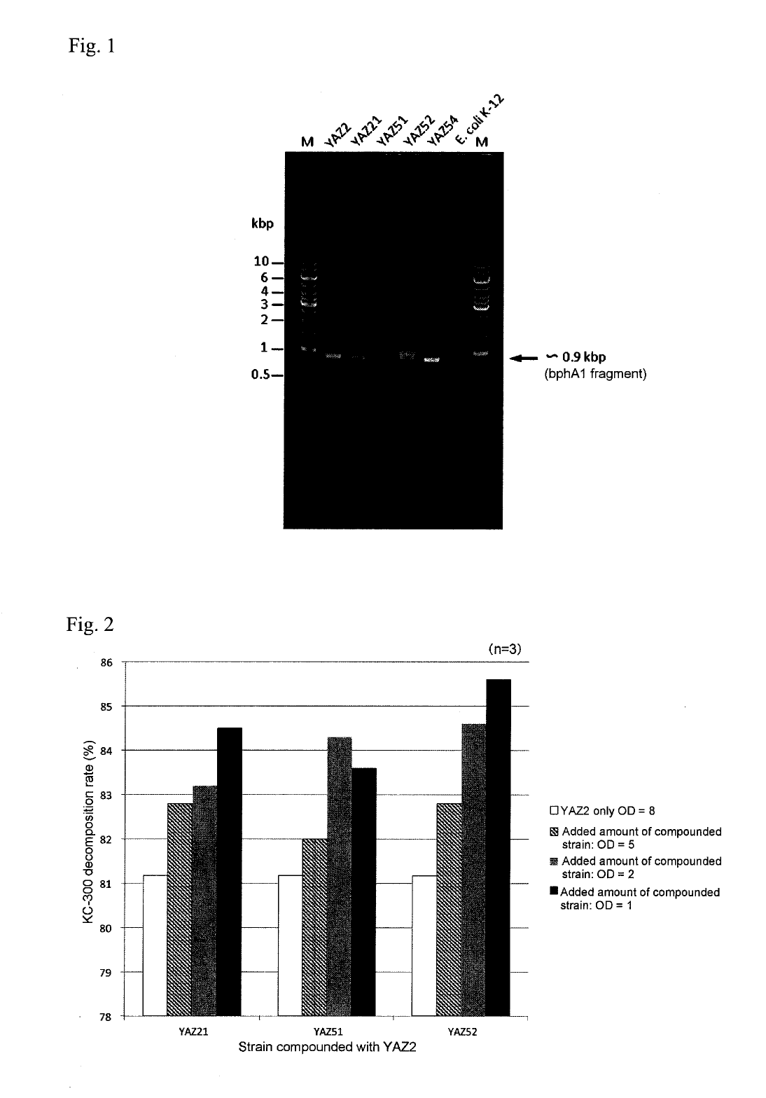

Degenerate primers were prepared by selecting several highly preserved regions (such as Asn-Gln/Ser-Cys-Arg/Ser-His-Arg-Gly-Met (SEQ ID NO: 1) or Glu-Gln-Asp-Asp-Gly/Thr-Glu-Asn (SEQ ID NO: 2)) based on a comparison of the amino acid sequences of the BphA1 (biphenyl-2,3-dioxygenase α-subunit) of known PCB-decomposing bacteria consisting of Burkholderia xenovorans strain LB400, Pseudomonas pseudoalcaligenes strain KF707, Acidovorax sp. strain KKS102, Rhodococcus jostii strain RHA1, Rhodococcus erythropolis and Bacillus sp. strain JF8. Microbial cells centrifugally harvested from 0.1 ml to 1.2 ml of culture broth obtained by culturing each of the microbial strains of Comamonas testosteroni strain YAZ2, Achromobacter sp. strain YAZ52, Pseudomonas sp. strain YAZ51, Rhodococcus sp. strain YAZ54 and Stenotrophomonas sp./Achromobacter sp. symbiotic strain YAZ21 to an OD₆₆₀ of 0.6 to 1.0 were suspended in a suitable amount of TE buffer (10 mM Tris-HCl, 1 mM EDTA, pH 8.0), and after heating for 15 to 20 minutes at 80° C. to 100° C., the centrifuged supernatants were used as thermal extracts of each microbial strain. PCR was then carried out using these as templates and using the prepared degenerate primers under reaction conditions consisting of 3 minutes at 94° C. followed by 35 cycles of 30 seconds at 94° C., 30 seconds at 58° C. to 60° C. and 1 minute at 72° C., and ending with 2 minutes at 72° C. In addition, the same reaction was carried out using a thermal extract containing the genome of Escherichia coli strain K-12 as a negative control not containing BphA1 gene. As a result, the amplification product of an approximately 900 bp BphA1 fragment was detected in all microbial strains, excluding Escherichia coli, as predicted, thereby confirming that these microbial strains have BphA1 gene (FIG. 1).

### Example 3

### Decomposition of Polychlorinated Biphenyls by Compounding Microorganisms

A required amount of a microbial composition obtained by adding Pseudomonas sp. strain YAZ51 or Achromobacter sp. strain YAZ52 to Comamonas testosteroni strain YAZ2 and compound therewith was preliminarily weighed out and added to 20 mM phosphate buffer solution to obtain a composition solution. The final concentration of the composition solution can be determined by measuring turbidity (OD₆₆₀), or the composition solution can be finely adjusted to a suitable concentration with 20 mM phosphate buffer solution. Moreover, a commercial polychlorinated biphenyl mixture in the form of Kanechlor KC-300 was added to the composition solution followed by aerating by inverting for 25 hours at a temperature of 30° C. to decompose PCBs.

TABLE 4

| | | | Complementary strain | | | |
|---|---|---|---|---|---|---|
| | | | YAZ51 | | YAZ52 | |
| | | | OD = | OD = | OD = | OD = |
| Main strain | | | 5 | 7 | 5 | 7 |
| | | Decomposition rate | 76.1 | — | 77.8 | — |
| YAZ2 | OD = 8 | 80.4 | — | 80.2 | — | 82.0 |
| | OD = 10 | — | 82.0 | — | 83.7 | — |

Decomposition rates (%) are shown after rounding to the second decimal place.

In Table 4 above, the microbial composite compositions demonstrated prominent improvement of decomposition rate in comparison with the case of only using a single microorganism. On the other hand, a high decomposition effect was demonstrated particularly in the case of making the incorporated ratio of Achromobacter sp. strain YAZ52 to Comamonas testosteroni strain YAZ2 to be from 1.1:1 to 2:1 in Table 3, although the mechanism behind this is not clear. This revolutionary result is thought to be due to the substrate specificity (referring to polychlorinated biphenyls in this case) of catabolic enzymes of polychlorinated biphenyls characteristically produced species-dependently by the microorganisms, and is not simply due to an increase in catabolic enzymes of polychlorinated biphenyls produced by the microorganisms as a result of compounding those microorganisms.

### Example 4

### Inhibitory Action on Polychlorinated Biphenyl Decomposition Attributable to Compounding Microorganisms

The results of investigating the decomposition activity on a commercial polychlorinated biphenyl mixture such as Kanechlor KC-300 of a composite composition obtained by incorporating an equal number of microbial cells of Rhodococcus sp. strain YAZ54 with microbial cells of Comamonas testosteroni strain YAZ2 using the same method as that described in Example 3 are shown in the following Table 5.

TABLE 5

| | Complementary strain | YAZ54 |
|---|---|---|
| Main strain | Decomposition rate (%) | 48.5 |
| YAZ2 | 78.7 | 70.1 |

(n = 3)

Decomposition rates (%) are shown after rounding to the second decimal place.

In Table 5 above, a composite composition obtained by incorporating an equal number of microbial cells of Rhodococcus sp. strain YAZ54 with microbial cells of Comamonas testosteroni strain YAZ2 demonstrated a remarkable decrease in the decomposition rate of polychlorinated biphenyls in comparison with the use of Comamonas testosteroni strain YAZ2 alone. Namely, decomposition activity with respect to polychlorinated biphenyls was shown to be able to be controlled by adjusting the microbial species or amount thereof.

### Example 5

### Decomposition Properties of Polychlorinated Biphenyls Attributable to Compounding Microorganisms

The results of investigating the polychlorinated biphenyl isomer decomposition properties of microbial strains listed in examples of the present invention using a commercial polychlorinated biphenyl mixture such as Kanechlor KC-300 according to the same method as that described in Examples 3 and 4 are shown in the following Table 6.

TABLE 6

| | | | | Isomer decomposition rates | | | | | | |
|---|---|---|---|---|---|---|---|---|---|---|
| | | | | | | YAZ2 + | | YAZ2 + | | YAZ2 + |
| | | | Abundance | YAZ2 | YAZ21 | YAZ21 | YAZ51 | YAZ51 | YAZ52 | YAZ52 |
| Chromatogram | | | ratio in | (O.D. = | (O.D. = | (O.D = | (O.D. = | (O.D. = | (O.D. = | (O.D = |
| peak no. | IUPAC No. | PCB isomer | KC-300 | 8) | 5) | 8 + 1) | 5) | 8 + 1) | 5) | 8 + 1) |
| 1 | #4. #10 | 22′. 26 | 1.06 | 100.0 | 100.0 | 100.0 | 100.0 | 100.0 | 100.0 | 100.0 |
| 2 | #7. #9 | 24. 25 | 0.15 | 100.0 | 100.0 | 100.0 | 100.0 | 100.0 | 100.0 | 100.0 |
| 3 | #6 | 23′ | 0.47 | 100.0 | 100.0 | 100.0 | 100.0 | 100.0 | 100.0 | 100.0 |
| 4 | #5. #8 | 23. 24′ | 4.68 | 100.0 | 100.0 | 100.0 | 100.0 | 100.0 | 100.0 | 100.0 |
| 5 | #19 | 22′6 | 0.54 | 0.0 | 0.0 | 0.1 | 12.7 | 0.0 | 0.0 | 0.0 |
| 6 | #12. #13 | 34. 34′ | 0.03 | 100.0 | 100.0 | 100.0 | 100.0 | 100.0 | 100.0 | 100.0 |
| 7 | #18 | 22′5 | 7.71 | 89.3 | 100.0 | 100.0 | 100.0 | 100.0 | 100.0 | 100.0 |
| 8 | #15. #17 | 44′. 22′4 | 5.79 | 100.0 | 100.0 | 100.0 | 100.0 | 100.0 | 100.0 | 100.0 |
| 9 | #24. #27 | 236. 23′6 | 0.58 | 100.0 | 100.0 | 100.0 | 52.7 | 100.0 | 70.5 | 100.0 |
| 10 | #16. #32 | 22′3. 24′6 | 5.76 | 88.2 | 58.4 | 84.6 | 66.6 | 83.7 | 54.7 | 84.9 |
| 12 | #29. #54 | 245. 22′66′ | 0.09 | 100.0 | 100.0 | 100.0 | 100.0 | 100.0 | 100.0 | 100.0 |
| 13 | #26 | 23′5 | 1.26 | 100.0 | 100.0 | 100.0 | 100.0 | 100.0 | 100.0 | 100.0 |
| 14 | #25 | 23′4 | 0.61 | 100.0 | 100.0 | 100.0 | 100.0 | 100.0 | 100.0 | 100.0 |
| 15, 16 | #31. #28 | 24′5. 244′ | 21.84 | 100.0 | 94.2 | 100.0 | 98.2 | 100.0 | 100.0 | 100.0 |
| 17 | #20. #33. | 233′. 23′4′. | 8.16 | 93.9 | 93.5 | 94.5 | 94.7 | 93.3 | 92.6 | 93.4 |
| | #53 | 22′56′ | | | | | | | | |
| 18 | #22. #51 | 234′. 22′46′ | 3.86 | 95.7 | 79.2 | 95.5 | 92.3 | 94.7 | 95.2 | 96.0 |
| 19 | #45 | 22′36 | 0.72 | 15.7 | 1.4 | 12.5 | 30.3 | 16.1 | 7.0 | 17.4 |
| 20 | #46 | 22′36′ | 0.21 | 12.3 | 3.4 | 11.5 | 45.9 | 14.7 | 38.2 | 22.2 |
| 21 | #52 | 22′55′ | 2.96 | 14.8 | 8.6 | 18.1 | 28.7 | 11.5 | 22.8 | 18.1 |
| 22 | #49 | 22′45′ | 3.00 | 19.2 | 4.1 | 21.1 | 23.5 | 17.5 | 1.1 | 18.9 |
| 23 | #47. #48 | 22′44′. 22′45 | 2.66 | 26.1 | 46.5 | 47.8 | 60.0 | 47.4 | 50.1 | 59.7 |
| 24 | #35 | 33′4 | 0.07 | 100.0 | 100.0 | 100.0 | 100.0 | 100.0 | 100.0 | 100.0 |
| 25 | #44 | 22′35′ | 2.81 | 5.8 | 95.7 | 82.8 | 99.4 | 86.5 | 100.0 | 100.0 |
| 26 | #59. #37. | 233′6. 344′. | 4.72 | 98.7 | 40.7 | 94.0 | 61.3 | 94.2 | 95.2 | 96.4 |
| | #42 | 22′34′ | | | | | | | | |
| 27 | #41. #64. | 22′34. 234′6. | 3.82 | 33.8 | 2.9 | 31.0 | 26.1 | 28.3 | 6.3 | 33.4 |
| | #71 | 23′4′6 | | | | | | | | |
| 29 | #40. #57 | 22′33′. 233′5 | 0.52 | 100.0 | 8.5 | 100.0 | 29.9 | 100.0 | 16.2 | 100.0 |
| 30 | #67 | 23′45 | 0.16 | 100.0 | 100.0 | 100.0 | 100.0 | 100.0 | 100.0 | 100.0 |
| 31 | #63 | 244′5 | 0.11 | 100.0 | 0.0 | 100.0 | 30.7 | 100.0 | 10.6 | 100.0 |
| 32 | #74 | 244′5 | 2.22 | 100.0 | 33.6 | 100.0 | 57.1 | 100.0 | 93.8 | 100.0 |
| 33 | #70 | 23′4′5 | 3.42 | 80.6 | 52.6 | 80.2 | 70.7 | 82.8 | 98.2 | 92.1 |
| 34 | #66. #95. | 23′44′. 22′35′6. | 4.48 | 93.0 | 19.9 | 92.5 | 42.8 | 92.8 | 69.1 | 93.1 |
| | #102 | 22′456′ | | | | | | | | |
| 35 | #55. #91 | 233′4. 22′34′6 | 0.11 | 72.4 | 0.3 | 42.0 | 8.6 | 64.0 | 36.9 | 70.1 |
| 36 | #56. #60. | 233′4′. 2344′. | 2.91 | 99.4 | 25.8 | 100.0 | 45.0 | 99.0 | 55.2 | 99.1 |
| | #92 | 22′355′ | | | | | | | | |
| 37 | #84. #90. | 22′33′6. 22′34′5. | 0.62 | 30.0 | 8.0 | 33.7 | 29.3 | 19.2 | 8.8 | 33.1 |
| | #101 | 22′455′ | | | | | | | | |
| 38 | #99 | 22′44′5 | 0.27 | 30.8 | 6.3 | 24.3 | 25.7 | 36.2 | 5.9 | 34.0 |
| 41 | #97 | 22′34′5′ | 0.17 | 25.1 | 9.6 | 22.3 | 57.3 | 43.3 | 56.2 | 30.4 |
| 42 | #87 | 22′345′ | 0.24 | 26.9 | 0.0 | 9.1 | 20.1 | 23.3 | 6.1 | 24.1 |
| 43 | #85 | 22′344′ | 0.13 | 21.0 | 0.0 | 0.0 | 32.7 | 18.2 | 12.4 | 22.4 |
| 45 | #77. #110 | 33′44′. 233′4′6 | 0.58 | 46.4 | 0.0 | 40.5 | 18.3 | 47.0 | 6.5 | 47.3 |
| 50 | #118 | 23′44′5 | 0.38 | 56.0 | 0.0 | 52.6 | 37.2 | 28.4 | 30.1 | 52.5 |
| 54 | #105. #132 | 233′44′. 22′33′46′ | 0.22 | 100.0 | 0.0 | 100.0 | 32.6 | 100.0 | 0.0 | 100.0 |
| | | KC-300 decom- | | 81.1 | 67.5 | 84.6 | 76.1 | 83.6 | 77.8 | 85.6 |
| | | position rate | | | | | | | | |

(n = 3)
unit (%)
Decomposition rates (%) are shown after rounding to the second decimal place.

In Table 6 above, decomposition rates indicate decomposition rates with respect to the abundance ratio of polychlorinated biphenyl isomers in Kanechlor KC-300. In the present example, the concentration of polychlorinated biphenyls was set to 5 ppm and the reaction conditions consisted of a temperature of 30° C. and duration of 25 hours. As a result, as indicated by the underlines in the table in particular, the case of preliminarily incorporating and compounding Pseudomonas sp. strain YAZ51 or Achromobacter sp. strain YAZ52 with Comamonas testosteroni strain YAZ2 clearly resulted in a remarkable improvement in decomposition rate with respect to 2,2′,4,4′-tetrachlorobiphenyl, 2,2′,4,5-tetrachlorobiphenyl and 2,2′,3,5′-tetrachlorobiphenyl present in the Kanechlor KC-300 in comparison with the case of Comamonas testosteroni strain YAZ2 alone. Namely, decomposition rates against polychlorinated biphenyl isomers were shown to differ according to differences in microbial species.

### Example 6

### Correlation Between Changes in Formulation of Microbial Composite Composition and Polychlorinated Biphenyl Decomposition

The results of preparing compositions having different incorporation ratios of microbial strains listed as examples in the present invention prepared using a commercial polychlorinated biphenyl mixture such as Kanechlor KC-300 according to the same method as that of Examples 3, 4 and 5, and investigating changes in the decomposition rates of polychlorinated biphenyls, are shown in FIG. 2.

In FIG. 2, as a result of setting the microbial cell concentration of Comamonas testosteroni strain YAZ2 to a fixed concentration of OD₆₆₀=8 and preliminarily preparing a composite composition therewith while changing the microbial cell concentration of Pseudomonas sp. strain YAZ51 or Achromobacter sp. strain YAZ52 from OD₆₆₀=1 to OD₆₆₀=7 followed by reacting with polychlorinated biphenyls having a concentration of 5 ppm, the case of using a composite composition in which Comamonas testosteroni strain YAZ2 was incorporated and compounded so that the concentration of Achromobacter sp. strain YAZ2 was OD₆₆₀=1 demonstrated the most remarkable improvement in decomposition rate.

### Reference Example 1

### Construction of Biphenyl Dioxygenase Expression Plasmid of Burkholderia xenovorans Strain LB400

Biphenyl dioxygenase activity includes biphenyl-2,3-dioxygenase (to be referred to as 2,3-dioxygenase) activity and biphenyl-3,4-dioxygenase (to be referred to as 3,4-dioxygenase) activity. As a result of investigating the biphenyl dioxygenase of more than 200 strains of environmental microorganisms acquired in Japan, the present applicant determined that all have 2,3-dioxygenase activity. Accordingly, the inventors of the present invention thought that the acquisition of an enzyme having 3,4-dioxygenase activity is important for further improving PCB decomposition rate.

A plasmid for use in gene recombination was created for the purpose of acquiring an enzyme having 3,4-dioxygenase activity based on the reasons described above. A 2120 bp (SEQ ID NO: 3) or 1,600 bp (SEQ ID NO: 6) DNA sequence containing BphA1A2 or BphA3A4 of Burkholderia xenovorans strain LB400 was used for the gene serving as the motif. These DNA sequences were subjected to PCR carried out under reaction conditions consisting of an initial temperature of 94° C. for 3 minutes followed by 28 cycles of 30 seconds at 94° C., 30 seconds at 60° C. and 2 minutes at 68° C., and ending by reacting for 3 minutes at 68° C., by combining primers 1 and 2 or primers 3 and 4 indicated below using plasmid pUC57-bphA1A2(LB400) or plasmid pUC57-bphA3A4(LB400) as a template, which were respectively inserted into a cloning vector pUC57 (Thermo Fisher Scientific Inc.), using an artificial gene created by organic chemical synthesis.

PrimeSTAR HS DNA Polymerase (Takara Bio Inc.) is preferably used for the DNA polymerase required for PCR. The reason for this is that high-quality plasmids can be constructed by suppressing the occurrence of erroneous gene substitutions that can occur in the PCR reaction.

The sequences of the aforementioned primers 1 to 4 are as indicated below.

Primer 1: 5′-ATGCATTCTAGATATTTTTTCCGCCCTGCCAAG-3′
(underline: restriction enzyme XbaI recognition
sequence, SEQ ID NO: 9)
Primer 2: 5′-ATGCATCCATGGCGTGCTGGGCTAGAAGAACAT-3′
(underline: restriction enzyme NcoI recognition
sequence: SEQ ID NO: 10)
Primer 3: 5′-ATGCATCCATGGCCCAGGCGATTTAACCCTTTTA-3′
(underline: restriction enzyme NcoI recognition
sequence: SEQ ID NO: 11)
Primer 4: 5′-ATCGATCATATGGCGATCAATTCGGTTTGGC-3′
(underline: restriction enzyme NdeI recognition
sequence: SEQ ID NO: 12)

After cleaving DNA fragments containing BphA1A2 or BphA3A4 gene of strain LB400 obtained in the aforementioned PCR with XbaI and NcoI or NcoI and NdeI, respectively, the fragments were purified by gel extraction followed by insertion into the XbaI-NcoI or NcoI-NdeI cleavage site of plasmid vector pET-15b (Novagen Inc.), respectively.

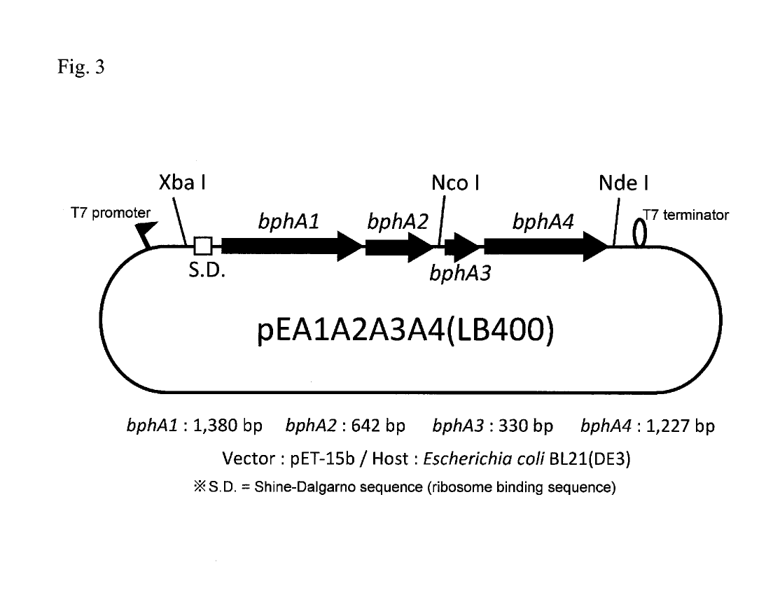

After confirming the absence of gene substitutions capable of occurring in the PCR reaction in each of the DNA sequences respectively inserted with the preliminarily prepared pET-15b-bpA1A2(LB400) and pET-15b-bphA3A4(LB400) plasmids, the NcoI-NdeI fragment containing bphA3A4(LB400) was cut out and inserted into the NcoI-NdeI site downstream from pET-15b-bphA1A2 to ultimately obtain strain LB400 BphA1A2A3A4 expression plasmid pEA1A2A3A4(LB400) (FIG. 3).

### Reference Example 2

### Confirmation of Expression of Enzyme Protein and PCB Decomposition Activity of Recombinant Escherichia coli Cells

Escherichia coli strain BL21(DE3) (Novagen Inc.) transformed with plasmid pEA1A2A3A4(LB400) prepared in the manner described above was cultured at 30° C. to a turbidity at a wavelength of 660 nm (OD₆₆₀) of 0.6 to 20 using 2×YT medium (1.6% tryptone, 1.0% yeast extract, 0.5% NaCl), LB medium (1.0% tryptone, 0.5% yeast extract, 0.5% NaCl) or TB medium (1.2% tryptone, 2.4% yeast extract, 0.8% glycerol, 54 mM K₂HPO₄, 16 mM KH₂PO₄), each containing 100 μg/ml of ampicillin. The use of an Erlenmeyer flask (Iwaki Glass Co., Ltd.), and preferably an Erlenmeyer flask with stirrer (Shibata Irika Co., Ltd.), makes it possible to impart the optimum dissolved oxygen concentration to the medium, allowing the obtaining of a microbial catalyst having biphenyl dioxygenase that demonstrates optimum PCB decomposition activity.

The volume of the actual culture broth when using these flasks was not more than 20% of the volume of the flasks. After adding an inducer in the form of isopropyl-β-thiogalactopyranoside (IPTG) to a final concentration of 0.05 mM to 0.5 mM at an appropriate time when the OD₆₆₀=0.6 to 20, culturing was further continued, and after washing the recombinant Escherichia coli, harvested by centrifugal separation 30 minutes to 5 hours later, with 20 mM sodium phosphate buffer (pH 7.5), the microbial cells were re-suspended in the same buffer. This suspension of recombinant Escherichia coli cells was adjusted a final concentration of OD₆₆₀=10, and a PCB solution, obtained by adjusting a commercial PCB mixture in the form of Kanechlor KC-300 (Kaneka Corp.) or similar standard containing Arochlor 1242 (Mitsubishi Monsanto Chemical Co.), and preferably Kanechor KC-300, was added to a final concentration of 5 ppm to 40 ppm followed by inverting at a speed of 50 rpm for 3 hours to 24 hours at a temperature of 30° C. to decompose the PCBs.

The amount of PCBs remaining in the solutions following the reaction was measured using a gas chromatograph-mass spectrometer (7890A/5075C, Agilent Technologies Inc., to be abbreviated as GC/MS). GC/MS analysis conditions were in accordance with “Control of Catalytic Reactions of Bacterial Preparations Highly Expressing Biphenyl Dioxygenase Using Ultrasonic Microbubbles (authors: Jiro Haratomi, Yasunori Makuta, Yumiko Takazuka, Katsunori Sano and Tokio Niikuni)” contained on pages 13 to 15 of the 2013 Proceedings of the 23rd Symposium on Environmental Engineering of the Japan Society of Mechanical Engineers. The analysis procedure consisted of adding a suitable amount of hydrochloric acid to the reaction solution to stop the reaction followed by adding an internal standard in the form of anthracene to a concentration of 1.6 ppm with respect to a PCB concentration of 5 ppm at the time the reaction started, and liquid-liquid extracting with an amount of ethyl acetate (special grade, Wako Pure Chemical Industries, Ltd.) equal to 1 to 2 times, and preferably 2 times, the amount of the reaction solution. Next, the organic solvent phase to which residual PCBs in the reaction solution had migrated was dehydrated with anhydrous sodium sulfate followed by suitably diluting with ethyl acetate corresponding to the detection limit sensitivity of the GC/MS and injecting into the GC/MS. The detection limit of the GC/MS at this time is preferably 10 ppt or lower.

PCB quantitative data was analyzed according to the procedure described below. The total peak area of PCBs present in the sample measured with the GC/MS was divided by the total area of the PCB standard measured in the same manner as a control, and this value was corrected using the area of anthracene used as an internal standard, thereby enabling calculation of the correct PCB concentration. Finally, PCB decomposition rate was derived using this PCB concentration. The equations used to calculate PCB concentration and PCB decomposition rate were as indicated below.

PCB concentration [ppm]=PCB concentration of control standard [ppm]×(total area of PCBs in sample/total PCB area of control)×(area of anthracene in control/area of anthracene in sample)

PCB decomposition rate [%]={(PCB concentration before Decomposition [ppm]−PCB concentration after decomposition [ppm])/PCB concentration before decomposition [ppm]}×100

The following provides an explanation of the results of the present test.

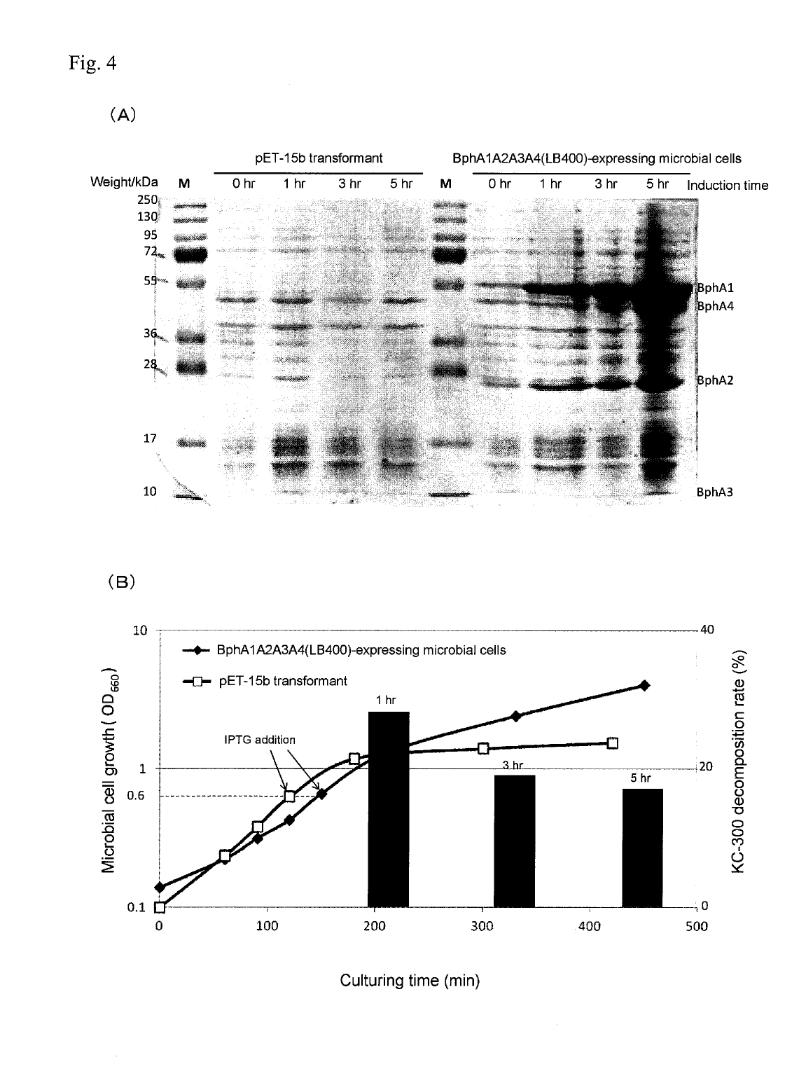

FIG. 4(A) indicates the results of using SDS-polyacrylamide gel electrophoresis (SDS-PAGE) at a gel concentration of 15% to analyze total protein of BphA1A2A3A4(LB400)-expressing microbial cells immediately prior to addition of IPTG to a final concentration of 0.5 mM at OD₆₆₀=0.6 (0 hours), and over time at 1, 3 and 5 hours after addition, by culturing Escherichia coli strain BL21(DE3) transformed with pEA1A2A3A4(LB400) or vector pET-15b only at a temperature of 30° C. in 2×YT medium containing ampicillin. Escherichia coli strain BL21(DE3) transformed with vector pET-15b only served as a control that did not express recombinant enzyme protein.

On the other hand, FIG. 4(B) indicates growth curves during addition of IPTG at a final concentration of 0.5 mM at OD₆₆₀=0.6, obtained by culturing Escherichia coli strain BL21(DE3) transformed with pEA1A2A3A4(LB400) or vector pET-15b only in 2×YT medium at a temperature of 30° C., and results indicating decomposition rates after subjecting a suspension of BphA1A2A3A4(LB400)-expressing microbial cells (concentration: OD₆₆₀=10) and Kanechlor KC-300 (5 ppm), harvested at 1, 3 and 5 hours after the same addition of IPTG, to a catalytic reaction for 24 hours. Escherichia coli strain BL21(DE3) transformed with vector pET-15b only served as a control in the same manner as FIG. 4(A).

FIG. 4(A) indicates that approximately 55 kDa, 45 kDa and 25 kDa proteins were expressed in BphA1A2A3A4(LB-400)-expressing microbial cells that were unable to be confirmed in the pET-15b transformant serving as control, or in other words, these can be inferred to correspond to BphA1 (molecular weight: approx. 51.5 k), BphA4 (approx. 43.0 k) and BphA2 (approx. 25.0 k), respectively. However, since the band of BphA3 having a molecular weight of approximately 12.0 k was located at the end of electrophoresis due to its small molecular weight under these electrophoresis conditions, its presence was unable to be definitively confirmed. On the basis of these results, expression levels of proteins corresponding to BphA1, BphA2 and BphA4 were confirmed to clearly increase with the passage of induction time following addition of IPTG.

Continuing, FIG. 4(B) indicates that, in contrast to decomposition activity not being detected in Escherichia coli cells transformed with vector pET-15b only, activity was detected in all microbial cells at 1, 3 and 5 hours after addition of IPTG in the case of strains expressing BphA1A2A3A4(LB400). Moreover, PCB decomposition activity of the microbial cells at 1 hour after addition of IPTG was 32.3% after 24 hours, demonstrating a higher decomposition rate than the microbial cells after 3 hours and 5 hours (demonstrating decomposition rates of 21.8% and 19.6%, respectively).

Although the above results clearly indicated a decrease in decomposition activity despite a time-based increase in the expression level of recombinant protein following addition of IPTG, the present applicants thought that, instead of PCB decomposition activity being dependent on recombinant protein expression level, it is important that the four types of subunit proteins consisting of BphA1 to BphA4 be expressed in the optimum balance for decomposition activity in terms of strongly supporting the present invention.

### Reference Example 3

### Detailed Study of Expression Induction Conditions of Recombinant Enzyme Proteins

The aforementioned Reference Example 2 indicated that the PCB decomposition activity of Escherichia coli cells expressing recombinant enzyme protein tended to differ according to differences in induction time following addition of IPTG. Therefore, the inventors of the present invention conducted a detailed study of the relationship between that induction time and PCB decomposition activity based on differences in the amount of IPTG added, in addition to a study of conditions for being able to produce a microbial catalyst having 3,4-biphenyl dioxygenase activity optimal for PCB decomposition.

Escherichia coli strain BL21(DE3) transformed with pEA1A2A3A4(LB400) were cultured at a temperature of 30° C. using 2×YT medium containing ampicillin in the same manner as the aforementioned Reference Example 2, followed by adding IPTG to a final concentration of 0.1 mM or 0.2 mM at OD₆₅₀=0.6. Culturing was further continued following addition of IPTG and microbial cells were harvested 30, 60, 90 and 120 minutes later. All of the harvested microbial cells were washed with sodium phosphate buffer, suspensions were prepared to a concentration of OD₆₆₀=10 with the same buffer, and the resulting suspensions were used in a 24-hour decomposition test using Kanechlor KC-300 at a concentration of 5 ppm.

The following provides an explanation of the results of the present test.

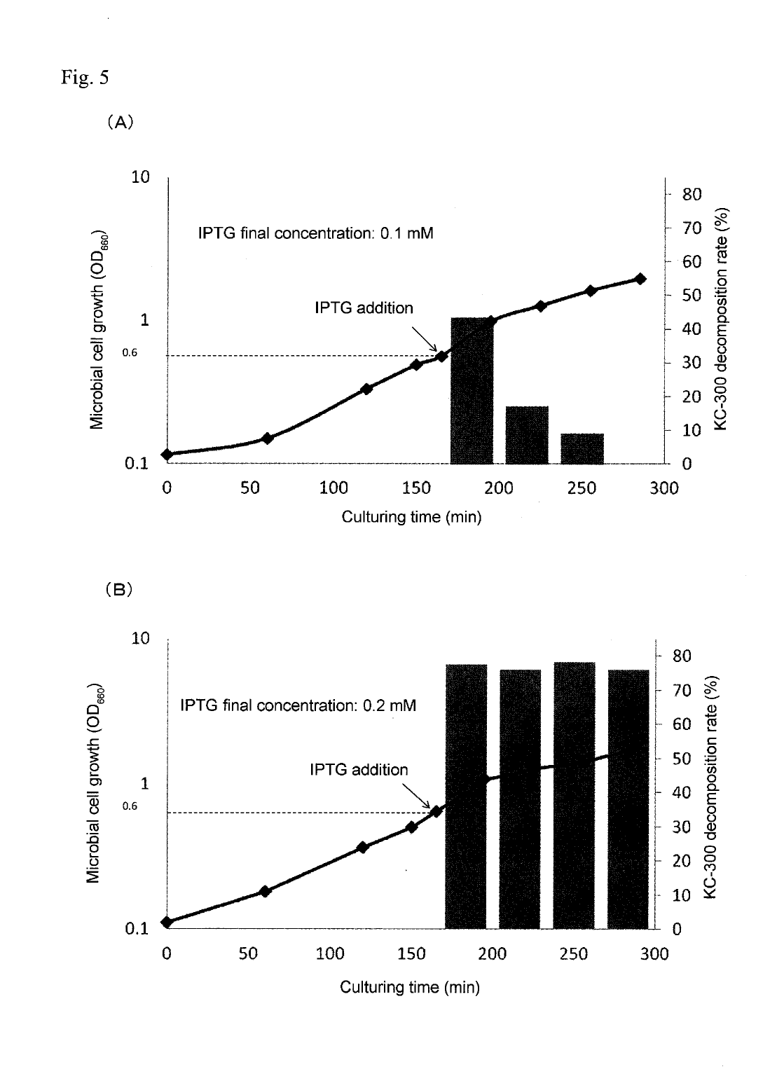

FIG. 5 indicates growth curves of a BphA1A2A3A4(LB400)-expressing strain when IPTG was added to a final concentration of 0.1 mM or 0.2 mM, and results obtained for PCB decomposition rate by microbial cells harvested over time.

As a result, a considerable difference in PCB decomposition activity was indicated between the case of an IPTG final concentration of 0.1 mM and 0.2 mM. Although there were no large differences observed in the growth curve of the microbial cells attributable to the difference in IPTG concentration, in contrast to PCB decomposition rates being extremely high at greater than 75% (77.4%, 76.0%, 78.2% and 75.9% after 30, 60, 90 and 120 minutes, respectively) regardless of induction time in the case of an IPTG concentration of 0.2 mM, in the case of an IPTG concentration of 0.1 mM, all PCB decomposition rates were below 50% (43.3%, 17.1%, 9.0% and 0% after 30, 60, 90 and 120 minutes, respectively). Accordingly, the optimum IPTG concentration was estimated to be 0.2 mM. Moreover, upon examination of FIG. 3, all of the microbial cells demonstrated stable and potent PCB decomposition activity from the start of induction to 120 minutes thereafter at the optimum IPTG concentration of 0.2 mM, with microbial cells after 90 minutes in particular exhibiting an extremely high decomposition rate of 78.2%.

These results clearly indicated that, in the case of making the final concentration of IPTG 0.2 mM and inducing expression for 90 minutes, a gene-recombinant biphenyl dioxygenase microbial catalyst can be produced that maintains extremely stable and potent PCB decomposition activity. The inventors of the present invention thought these induction conditions suggest a “state in which the four types of subunit proteins consisting of BphA1 to BphA4 are optimally expressed for PCB decomposition activity” as hypothesized in the aforementioned Example 2.

Since information relating to the optimum concentration and induction time of an inducer in the form of IPTG that allow the obtaining of highly active BphA1A2A3A4(LB400)-expressing microbial cells can be obtained based on the above results, a survey was also conducted on the optimum turbidity value when adding IPTG during growth of the microbial cells.

Escherichia coli strain BL21(DE3) transformed with pEA1A2A3A4(LB400) was cultured at 30° C. using 2×YT medium containing ampicillin using the same method as Reference Example 2. IPTG was added to a final concentration of 0.2 mM when OD₆₆₀ reached 0.6, 1.0 or 3.0, and after harvesting the microbial cells following an induction time of 90 minutes and washing with sodium phosphate buffer, the microbial cells were re-suspended in the same buffer. After adjusting the final concentration of the microbial cells to OD₆₆₀=10, Kanechlor KC-300 was added to a final concentration of 5 ppm and decomposed for 24 hours at a temperature of 30° C.

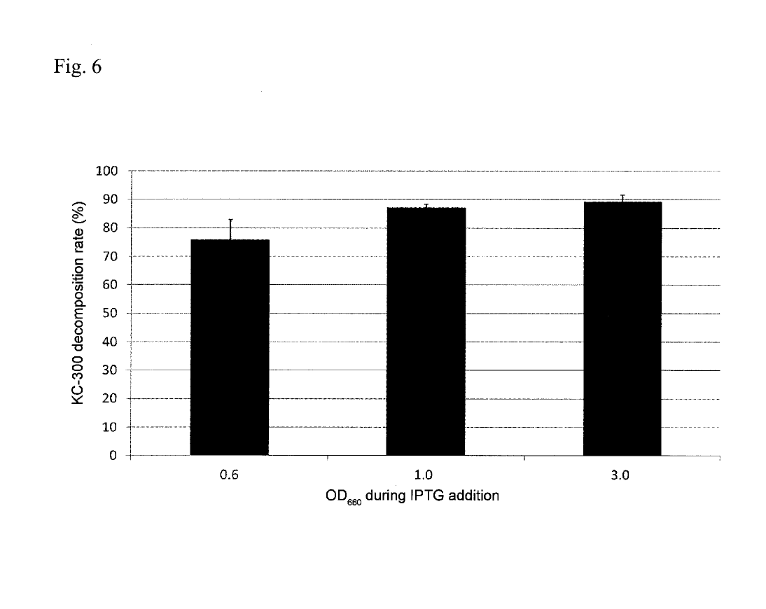

The amount of PCBs remaining following the present decomposition reaction test was quantified using the method described in the aforementioned Reference Example 2, and the results of analysis are shown in FIG. 6 and Table 7.

TABLE 7

| | Average | n |
|---|---|---|
| OD₆₆₀ during | decomposition rate | (number of |
| IPTG addition | (%) | times repeated) |
| 0.6 | 75.8 (±7.0) | 5 |
| 1.0 | 87.2 (±1.1) | 6 |
| 3.0 | 89.3 (±2.3) | 6 |

The following provides an explanation of the results.

According to FIG. 6 and Table 7, in any of the cases in which the growth of microbial cells to which IPTG had been added reached an OD₆₆₀ of 0.6, 1.0 or 3.0, microbial cells expressing BphA1A2A3A4(LB400) were determined to demonstrate stable and potent decomposition activity, and the resulting PCB decomposition rates were 75.8±7.0%, 87.2±1.1% and 89.3±2.3%, respectively.

On the basis of this result, induction conditions for obtaining a microbial catalyst in a “state in which the four types of subunit proteins consisting of BphA1 to BphA4 are optimally expressed for PCB decomposition activity” were thought to be dependent on IPTG concentration and induction time following addition of IPTG, and that differences in OD₆₆₀ values during addition of IPTG have hardly any effect.

In addition, from the viewpoint of producing this microbial catalyst in even larger volume, further examination of the aforementioned results indicated that the number of microbial cells that maintain a high level of PCB decomposition activity at completion of culturing increased approximately 4-fold when OD₆₆₀=1.0 and approximately 7-fold when OD₆₆₀=3.0 in comparison with the number of microbial cells obtained when IPTG was added at OD₆₆₀=0.6. Namely, the present invention was determined to comprise an extremely efficient industrial process capable of large-volume production of a microbial catalyst while maintaining the high level of PCB decomposition activity of BphA1A2A3A4(LB400)-expressing microbial cells by adding IPTG when the turbidity value during microbial growth is high.

### Reference Example 4

### Evaluation of PCB Isomer Decomposition Activity of Microbial Cells Expressing BphA1A2A3A4(LB400)

The 2,3-dioxygenase activity and 3,4-dioxygenase activity demonstrated by BphA1A2A3A4-expressing recombinant microbial cells of strain LB400 prepared in the aforementioned Reference Example 2 were confirmed. Comamonas testosteroni strain YAZ2 exhibiting 2,3-dioxygenase activity acquired in Example 1 was used for the control microbial cells used during confirmation.

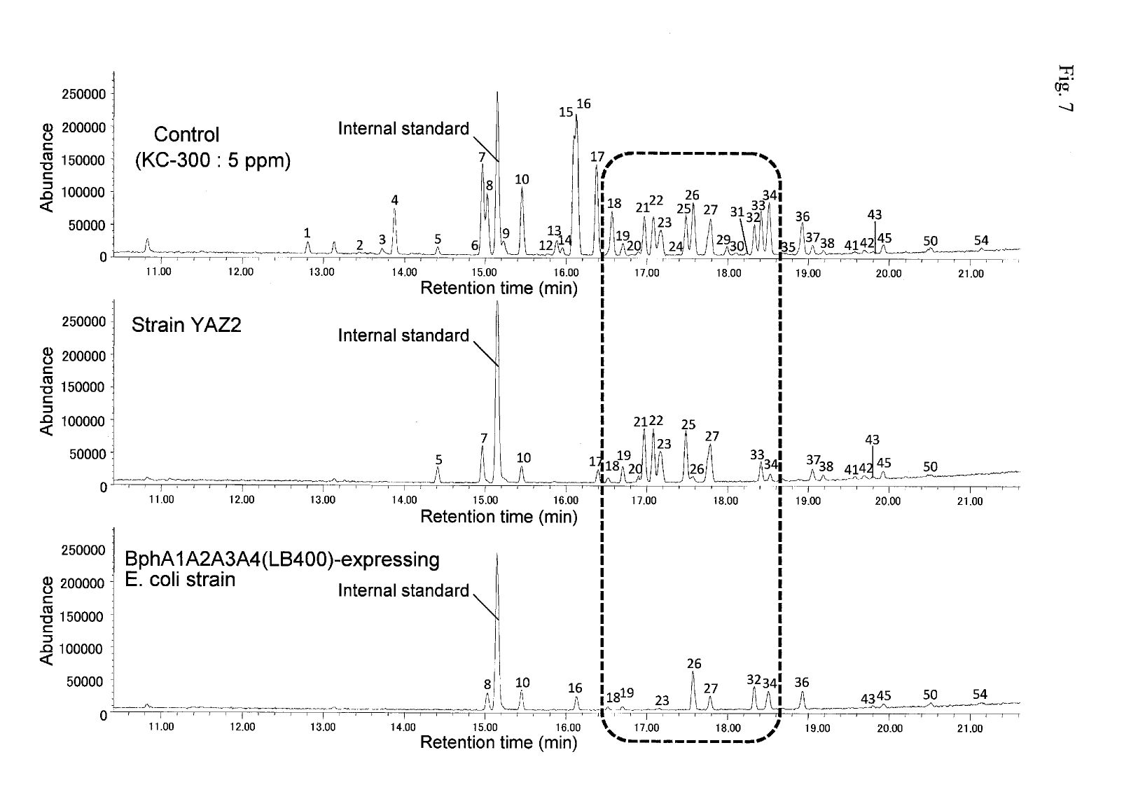

Preparation of the BphA1A2A3A4(LB400)-expressing microbial cells used in the test of the present reference example was carried out in the same manner as the aforementioned Reference Example 2. Expression induction conditions of the recombinant protein in particular consisted of making the final concentration of IPTG added 0.2 mM based on the results obtained in the aforementioned Reference Example 3 and harvesting the microbial cells 90 minutes after inducing expression. On the other hand, microbial cells obtained by culturing wild type Comamonas testosteroni strain YAZ2 in medium containing biphenyl was used for the microbial strain having 2,3-dioxygenase activity only, a required number of the microbial cells was washed with sodium phosphate buffer, and the cells were used after re-suspending in the same buffer. After adjusting the concentrations of each of the suspensions of these two types of microbial strains to a final concentration of OD₆₆₀=10, Kanechlor KC-300 was added to a concentration of 5 ppm and decomposed for 24 hours at a temperature of 30° C. PCBs remaining after the decomposition reaction were analyzed using the same method as the GC-MS method described in the aforementioned Reference Example 2, and the analysis results are shown in FIG. 7.

The following provides an explanation of the results.

According to FIG. 7, PCB isomers remaining after the reaction differed considerably between the microbial strain expressing BphA1A2A3A4(LB400) and strain YAZ2, and in contrast to strain YAZ2 demonstrating hardly any decomposition of PCB isomers contained in Kanechlor KC-300 consisting of 2,2′,3,6-tetrachlorobiphenyl (peak no. 19), 2,2′,5,5′-tetrachlorobiphenyl (peak no. 21), 2,2′,4,5′-tetrachlorobiphenyl (peak no. 22), 2,2′,4,4′-tetrachlorobiphenyl or 2,2′,4,5-tetrachlorobiphenyl (peak no. 23), the BphA1 A2A3A4(OLB400)-expressing strain completely decomposed all of these PCB isomers. On the other hand, in contrast to the BphA1A2A3A4(LB400)-expressing strain hardly demonstrating any decomposition of 2,4,4′,5-tetrachlorobiphenyl, strain YAZ2 nearly completely decomposed this isomer.

On the basis of this result, microbial cells expressing BphA1A2A3A4(LB400) capable of decomposing 2,2′,4,4′-tetrachlorobiphenyl were able to be confirmed to have 2,3-dioxygenase activity, and since they completely decomposed 2,2′,5,5′-tetrachlorobiphenyl, which adopts a chlorine-substituted structure that cannot be decomposed by 2,3-dioxygenase and a structure in which chlorine is not substituted at positions 3 and 4, they can be considered to also have 3,4-dioxygenase activity, thereby making it possible to acquire candidates of microbial catalysts having completely novel substrate specificity, which are not found in the wild types acquired in nature by the present applicant, by producing Escherichia coli strain BL21(DE3) transformed with the plasmid pEA1A2A3A4(LB400) in the present invention.

### Reference Example 5

### Decomposition of PCBs by Compounding Microbial Cells Expressing BphA1A2A3A4(LB400) and Comamonas testosteroni Strain YAZ2

PCB decomposition rate in the case of compounding the BphA1A2A3A4(LB400)-expressing microbial cells produced in the aforementioned Reference Example 2 with the Comamonas testosteroni strain YAZ2 acquired in Example 1 was compared with PCB decomposition rates in the case of each strain alone in an attempt to verify the significance of compounding. Moreover, a study was also made as to what type of effect a change in the compounding ratio of the BphA1A2A3A4(LB400)-expressing cells has on PCB decomposition rate.

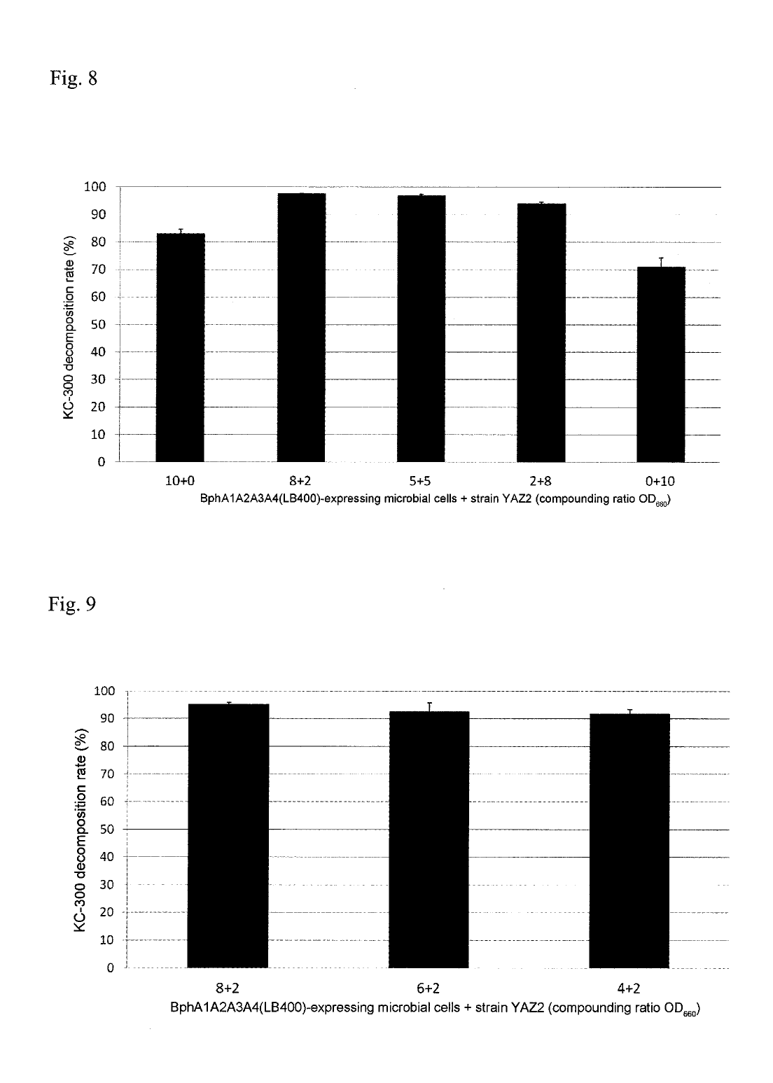

BphA1A2A3A4(LB400)-expressing cells and strain YAZ2 alone, as well as microbial catalysts, prepared at a compounding ratio of BphA1A2A3A4(LB400)-expressing microbial cells to strain YAZ2 adjusted to 8:2, 5:5 or 2:8 in terms of OD₆₆₀ turbidity using the same BphA1A2A3A4(LB400)-expressing microbial cells and Comamonas testosteroni strain YAZ2 as the aforementioned Reference Example 4, were allowed to undergo a catalytic reaction with Kanechlor KC-300 at a concentration of 5 ppm for 24 hours at a temperature of 30° C. PCBs remaining after the reaction were analyzed using the same method as the GC-MS method described in the aforementioned Example 2, and the analysis results are shown in FIG. 8 and Table 8.

TABLE 8

| BphA1A2A3A4(LB400)-expressing | Decomposition rate |
|---|---|
| microbial cells: Strain YAZ2 | (%) |
| 10:0 | 83.1 (±1.6) |
| 8:2 | 97.6 (±0.1) |
| 5:5 | 97.0 (±0.4) |
| 2:8 | 94.0 (±0.6) |
| 0:10 | 71.0 (±3.3) |

The following provides an explanation of the results.

According to FIG. 8 and Table 8, when a comparison is made of PCB decomposition rates for each microbial cell compounding ratio, in contrast to the decomposition rate in the case of BphA1A2A3A4(LB400)-expressing microbial cells alone being 83.1±1.6% and that in the case of strain YAZ2 alone being 71.0±3.3%, decomposition rates for all of the composite microbial catalysts exceeded 90% (97.6±0.1%, 97.0±0.4% and 94.0±0.6% when the OD₆₆₀ of the BphA1A2A3A4(LB400)-expressing microbial cells was 8, 5 and 2, respectively). This analysis was carried out 3 times (n=3).

This result indicated that a catalyst obtained by compounding microorganisms having different substrate specificities is more significant than when not compounding in order to efficiently decompose PCBs, and suggested that the optimum compounding ratio for that purpose is such that the number of BphA1A2A3A4(LB400)-expressing microbial cells tends to be greater than that of strain YAZ2 and that the OD₆₆₀ turbidity ratio is preferably 8:2.

A more detailed study was attempted regarding the aforementioned compounding ratio.

TABLE 9

| BphA1A2A3A4(LB400)-expressing | Decomposition rate |
|---|---|
| microbial cells: Strain YAZ2 | (%) |
| 8:2 | 95.2 (±0.7) |
| 6:2 | 92.7 (±3.1) |
| 4:2 | 91.9 (±1.5) |

In FIG. 9 and Table 9, a study was made of the optimum compounding ratio of BphA1A2A3A4(LB400)-expressing cells for decomposition of PCBs when the compounded amount of strain YAZ2 was fixed at OD₆₆₀=2. More specifically, the number of BphA1A2A3A4(LB400)-expressing cells was varied among OD₆₆₀=8, 6 and 4. As a result, the decomposition ratio was 95.2±0.7% in the case of OD₆₆₀=8, 92.7±3.1% in the case of OD₆₆₀=6 and 91.9±1.5% in the case of OD₆₆₀=4, indicating that PCB decomposition rate decreased as the number of BphA1A2A3A4(LB400)-expressing microbial cells decreased and that the highest decomposition rate was demonstrated in the case of OD₆₆₀=8. Based on GC-MS chromatogram data, the residual amounts of isomers containing 2,2′,6-trichlorobiphenyl, 2,2′,4,4′-tetrachlorobiphenyl, 2,2′,4,5-tetrachlorobiphenyl, 2,2′,3,4-tetrachlorobiphenyl, 2,3,4′,6-tetrachlorobiphenyl and 2,3′,4′,6-tetrachlorobiphenyl were confirmed to increase accompanying a decrease in the compounded number of BphA1A2A3A4(LB400)-expressing microbial cells. It is difficult to decompose these isomers with strain YAZ2. Accordingly, this phenomenon suggested that microbial cells expressing BphA1A2A3A4(LB400) have the ability to decompose more types of PCB isomers than strain YAZ2.

This result indicates the case in which a compounding ratio of a composite microbial catalyst such that the ratio of BphA1A2A3A4(LB400)-expressing cells to strain YAZ2 in terms of OD660 turbidity is 8:2 is optimal for decomposition of PCBs.

### Reference Example 6

### Test Using Compact PCB Decomposition Apparatus Equipped with Oxygen Microbubble Generation Mechanism

Decomposition efficiency with respect to various PCB isomers was verified using a compact decomposition apparatus equipped with an oxygen microbubble generation mechanism using a microbial catalyst obtained by compounding an Escherichia coli strain expressing BphA1A2A3A4(LB400) and wild type Comamonas testosteroni strain YU14-111, which express two types of dioxygenase having different PCB isomer decomposition properties. The compact decomposition apparatus used in the present example was the same as the apparatus described in FIGS. 3 and 4 of Japanese Patent Application No. 2013-141383.

Preparation of microbial cells expressing BphA1A2A3A4(LB400) was carried out in the same manner as the aforementioned Reference Example 4, the microbial cells were cultured at a temperature of 30° C. to OD₆₆₀=4.0 to 5.0, and preferably 5.0, using 2×YT medium containing ampicillin, and the cells were harvested 90 minutes after adding IPTG to a final concentration of 0.2 mM. The harvested microbial cells were washed with buffer and then used after re-suspending in the same buffer as that used for washing. On the other hand, preparation of Comamonas testosteroni strain YU14-111 was carried out by weighing out the required amount of a prepared preparation in the same manner as the method described in Japanese Unexamined Patent Publication No. 2013-179890 followed by washing with the same buffer as that described above and using after re-suspending in the same buffer.

In the present study, a compact decomposition apparatus equipped with a mechanism capable of generating microbubbles by a pressurization method was used for the compact decomposition apparatus capable of generating oxygen microbubbles. The following provides an explanation of reaction procedure.

First, sodium phosphate buffer having a dissolved oxygen concentration of 20 ppm or more and preferably 28 ppm or more preliminarily filled with oxygen microbubbles by pressurization was introduced into the PCB decomposition reaction tank equipped in the aforementioned compact decomposition apparatus. Next, a preparation, obtained by compounding Escherichia coli cells expressing BphA1A2A3A4(LB400) and wild type Comamonas testosteroni strain YU14-111 cells at a ratio of 19:1 to 12:8, and preferably 16:4, in terms of OD₆₆₀ turbidity, was added to the apparatus. Continuing, PCB-contaminated insulation oil (PCB final concentration: 40 ppm) and a surfactant in the form of Triton X-100 at a final concentration of 0.001% to 0.01%, and preferably 0.005%, were added followed by allowing the decomposition reaction to proceed using a final volume of reaction liquid of 1 L. The temperature of the reaction tank during the reaction was maintained at 30±2° C. The concentration of dissolved oxygen during the reaction was adjusted so as to maintain at a concentration of 20 ppm to 40 ppm, and preferably 28 ppm or more, by continuously or intermittently supplying oxygen gas so as to be added to the reaction tank in which the partial pressure had been increased in advance. Oxygen was added by aerating with oxygen gas from the bottom of the reaction tank or by using a sparger made of PTFE containing as many pores having a diameter of 1 micrometer or less as possible obtained by modifying oxygen microbubble filling ports provided on the lower side of the reaction tank. The reaction liquid was agitated in order to carry out the optimal reaction, namely to carry out dispersion of PCBs and composite microbial catalyst in the reaction liquid and the catalytic reaction optimally. Agitation force equivalent to 40 rpm was imparted while using physical agitation force generated by stirring blades or lifting force generated by oxygen aeration and oxygen microbubbles.

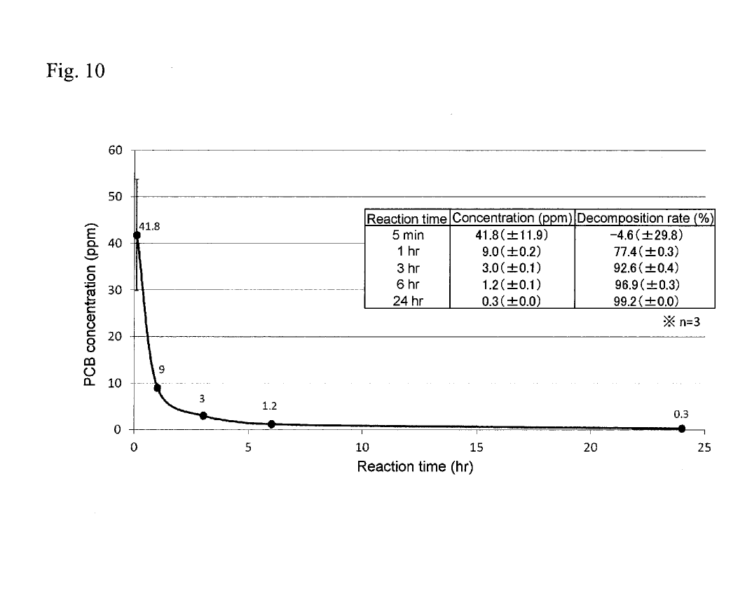

Portions of the reaction solution were sampled at 5 minutes, 1 hour, 3 hours, 6 hours and 24 hours after initiating contact between the PCBs and compound microbial catalyst, and time-based changes in the residual amount of PCBs were measured using GC-MS in the same manner as Reference Example 2, the results of which are shown in FIG. 10 and Table 10.

TABLE 10

| | | |
|---|---|---|
| | PCB | PCB |
| | concentration | decomposition rate |
| Reaction time | (ppm) | (%) |

| | | | |
|---|---|---|---|
| 5 | minutes | 41.8 (±11.9) | −4.6 (±29.8) |
| 1 | hour | 9.0 (±0.2) | 77.4 (±0.3) |
| 3 | hours | 3.0 (±0.1) | 92.6 (±0.4) |
| 6 | hours | 1.2 (±0.1) | 96.9 (±0.3) |
| 24 | hours | 0.3 (±0.0) | 99.2 (±0.0) |

According to the measurement results, PCBs initially added at 40 ppm rapidly decreased to 9.0±0.2 ppm 1 hour after starting the reaction, and further decreased to 3.0±0.1 ppm after 3 hours. In terms of decomposition rate, an extremely high decomposition rate of 92.6±0.4% was demonstrated. Moreover, decomposition had proceeded to a PCB level of 1.2±0.1 ppm (decomposition rate: 96.9±0.3%) 6 hours after starting the reaction, and stable decomposition of PCBs down to a PCB level of 0.3±0.0 ppm (decomposition rate: 99.2±0.0%) was demonstrated at 24 hours after starting the reaction, thereby demonstrating decomposition performance having extremely high activity and high efficiency that is below the accepted level of 0.5 ppm stipulated by the Ministry of the Environment. The aforementioned analysis was repeated three times (n=3).

### INDUSTRIAL APPLICABILITY

The present invention has remarkably high industrial utility value in that it further improves the PCB decomposition effects of individual PCB-decomposing microorganisms. For example, in the case of cleaning for the purpose of detoxifying capacitors and transformers containing and contaminated with polychlorinated biphenyls, by injecting a cleaning solvent containing the composition of the present invention into the capacitor and using it to clean the inside thereof, polychlorinated biphenyls contained therein are thought to be able to be decomposed or detoxified. In addition, capacitors and transformers containing polychlorinated biphenyls are thought to be able to be similarly decomposed and detoxified by adding the composition of the present invention to a cleaning solvent used to clean them. In this manner, it is self-evident that the composition of the present invention is effective for decomposing or detoxifying contaminants or their waste products, including equipment contaminated by polychlorinated biphenyls, located throughout Japan or overseas.

### Sequence Listing Free Text

SEQ ID NO: 1: Amino acid sequence of highly preserved region in BphA1 amino acid sequence of various PCB-decomposing microorganisms.

SEQ ID NO: 2: Amino acid sequence of another highly preserved region in BphA1 amino acid sequence of various PCB-decomposing microorganisms.

SEQ ID NO: 3: Base sequence of DNA encoding BphA1 and BphA2 derived from Burkholderia xenovorans strain LB400.

SEQ ID NO 4: Amino acid sequence of BphA1 derived from Burkholderia xenovorans strain LB400.

SEQ ID NO: 5: Amino acid sequence of BphA2 derived from Burkholderia xenovorans strain LB400.

SEQ ID NO: 6: Base sequence of DNA encoding BphA3 and BphA4 derived from Burkholderia xenovorans strain LB400.

SEQ ID NO: 7: Amino acid sequence of BphA3 derived from Burkholderia xenovorans strain LB400.

SEQ ID NO: 8: Amino acid sequence of BphA4 Burkholderia xenovorans strain LB400.

SEQ ID NO: 9: Base sequence of PCR primer 1

SEQ ID NO: 10: Base sequence of PCR primer 2

SEQ ID NO: 11: Base sequence of PCR primer 3

SEQ ID NO: 12: Base sequence of PCR primer 4

1. A method for producing a polychlorinated biphenyl-decomposing composition: comprising, a step for respectively culturing at least one main microbial strain belonging to the Comamonas species and having biphenyl dioxygenase, and at least one complementary microbial strain selected from the group consisting of Pseudomonas species, Achromobacter species, Rhodococcus species and Stenotrophomonas species and having biphenyl dioxygenase, by aeration-agitation culturing in medium containing biphenyl for the carbon source thereof, and a step for mixing at least two types of microbial cells recovered from each of the culture media.

2. The production method according to claim 1, further comprising a step for respectively adding an excipient to the at least two types of microbial cells and drying, and a step for compounding the microbial cells.

3. The production method according to claim 1, wherein the mixing ratio of the main microbial strain and the complementary microbial strain is a ratio of 0.5 to 9.9 of the complementary microbial strain to 10 of the main microbial strain in terms of the number of microbial cells converted based on turbidity of the culture media.

4. The production method according to claim 1, wherein the main microbial strain belongs to Comamonas testosteroni, the complementary microbial strain is one or two or more polychlorinated biphenyl-decomposing microorganisms belonging to Achromobacter species, and the mixing ratio of the main microbial strain and complementary microbial strain is a ratio of 1 to 6 of the complementary microbial strain to 10 of the main microbial strain in terms of the number of microbial cells converted based on turbidity of the culture media.

5. The production method according to claim 1, wherein the main microbial strain includes strain YU14-111 (Reference No.: NITE BP-01215), strain YAZ1 and/or strain YAZ2 belonging to Comamonas testosteroni.

6. The production method according to claim 1, wherein the complementary microbial strain includes Pseudomonas sp. strain YAZ51, Achromobacter sp. strain YAZ52, Rhodococcus sp. strain YAZ54 and/or Stenotrophomonas sp./Achromobacter sp. symbiotic strain YAZ21.

7. A polychlorinated biphenyl-decomposing composition produced according to the method according to claim 1.

8. A polychlorinated biphenyl-decomposing composition, comprising: at least one main microbial strain belonging to Comamonas species and having biphenyl dioxygenase, and at least one complementary microbial strain selected from the group consisting of Pseudomonas species, Achromobacter species, Rhodococcus species and Stenotrophomonas species and having biphenyl dioxygenase, at a ratio of 0.5 to 9.9 of the complementary microbial strain to 10 of the main microbial strain in terms of the number of microbial cells converted based on the turbidity of the culture media.

9. The polychlorinated biphenyl-decomposing composition according to claim 8, further comprising microbial cells expressing a biphenyl dioxygenase complex having biphenyl-3,4-dioxygenase activity against at least one type of polychlorinated biphenyl.

10. The polychlorinated biphenyl-decomposing composition according to claim 9, wherein the biphenyl dioxygenase complex is derived from Burkholderia sp. strain LB400.

11. The polychlorinated biphenyl-decomposing composition according to claim 10, wherein the biphenyl dioxygenase complex contains a protein composed of the amino acid sequence indicated in SEQ ID NO: 4, 5, 7 and 8, or contains a homologous protein having sequence homology of 90% or more with each of the amino acid sequences, and a complex thereof has polychlorinated biphenyl decomposition activity.

12. A method for decomposing polychlorinated biphenyls, comprising:
a step for mixing and emulsifying an oily component containing polychlorinated biphenyls, the composition according to claim 7, and depending on the case, an aqueous medium containing a surfactant, and
a step for aerating and agitating the aforementioned emulsion.

13. The method for decomposing polychlorinated biphenyls according to claim 12, further comprising supplying microbubbles to the aqueous medium and/or the emulsion.
